(19) United States
(12) Patent Application Publication
(10) Pub. No.: US 2011/0196017 A1
(43) Pub. Date: Aug. 11, 2011

(54) MICRO-RNA THAT PROMOTES VASCULAR INTEGRITY AND USES THEREOF

(76) Inventors: Eric N. Olson, Dallas, TX (US); Shusheng Wang, Dallas, TX (US)

(21) Appl. No.: 13/058,801

(22) PCT Filed: Aug. 11, 2009
(86) PCT No.: PCT/US09/53409

§ 371 Date: Apr. 28, 2011

Related U.S. Application Data
(60) Provisional application No. 61/087,886, filed on Aug. 11, 2008.

(51) Int. Cl. A61K 31/7088; A61K 48/00; A61P 9/00; A61P 35/00; A61P 9/10

(52) U.S. Cl. 514/44 A; 514/44 R

(57) ABSTRACT

The present invention relates to the identification of a microRNA, designated miR-126, that is a regulator of vascular integrity in endothelial cells. This endothelial cell-restricted microRNA mediates developmental angiogenesis in vivo, and targeted deletion of miR-126 in mice causes leaky vessels, hemorrhaging, and partial embryonic lethality, due to a loss of vascular integrity and defects in endothelial cell proliferation, migration, and angiogenesis. These vascular abnormalities resemble the consequences of diminished signaling by angiogenic growth factors, such as VEGF and FGF. These findings have important therapeutic implications for a variety of disorders involving abnormal angiogenesis and vascular leakage. Methods of treating disease states characterized by ischemia, vascular damage, and pathologic neovascularization by modulating miR-126 function are disclosed.

This application claims priority to U.S. Provisional Application Ser. No. 61/087,886 filed on Aug. 11, 2008, the entire disclosure of which is specifically incorporated herein by reference in its entirety.

This invention was made with grant support under grant no. HL53351-06 from the National Institutes of Health. The government has certain rights in the invention.

### BACKGROUND OF THE INVENTION

1. Field of the Invention

The present invention relates generally to the fields of cardiology, pathology and molecular biology. More particularly, it concerns gene regulation and cellular physiology in endothelial cells and muscle cells, by miR-126. This miRNA is plays an important role in maintaining vascular integrity and promoting vascular repair, and it can thus be used as a target for modulators in the treatment of disease.

2. Description of Related Art

Endothelial cells (ECs) line the internal surfaces of vascular structures and play essential roles in vascular development, function, and disease (Carmeliet, 2003). During blood vessel formation, known as vasculogenesis, ECs proliferate, migrate, and associate to form a primitive vascular labyrinth that serves as a scaffold for recruitment of smooth muscle cells. Subsequent sprouting of vessels, through angiogenesis, allows for further expansion of the vascular system and tissue vascularization.

Numerous peptide growth factors promote angiogenesis by enhancing EC migration, proliferation, survival, and cell-cell interactions. VEGF and FGF, the most potent angiogenic growth factors, are required for neoangiogenesis during embryogenesis and adulthood (Cross and Claesson-Welsh, 2001). Binding of these factors to their cell surface receptors activates the MAP kinase pathway, which promotes angiogenic growth and maturation. Conversely, inhibition of MAP kinase signaling diminishes angiogenesis (Eliceiri et al., 1998; Giroux et al., 1999; Hood et al., 2002), and has been advanced as an anti-angiogenic therapy (Hood et al., 2002; Panka et al., 2006).

Despite these advances, an incomplete understanding of the cell regulatory mechanisms relating to angiogenesis and vascular integrity and repair remains. Thus, a more complete understanding of these processes would substantially aid in efforts to identify agents can regulate these functions in vivo, e.g., in the treatment of disease.

### SUMMARY OF THE INVENTION

Thus, in accordance with the present invention, there is provided a method of promoting vascular integrity and/or endothelial repair comprising administering to a subject at risk of or suffering from vascular damage an agonist of miR-126 function. The subject suffering from vascular damage may be affected by vascular damage in a cardiac tissue, such as an ischemic event, including one comprising an infarct, ischemia-reperfusion injury or arterial stenosis. The vascular damage may be to a non-cardiac tissue, and may include trauma, or vascular leakage. Alternatively, where the subject is at risk of vascular damage, the subject may suffer from hypertension, cardiac hypetrophy, osteoporosis, neurodegeneration, fibrosis or respiratory distress. The method may further comprise administering to said subject a secondary therapy.

The subject may be a non-human animal or a human. The agonist may be miR-126 or a mimetic of miR-126. The agonist also may be an expression vector that comprises a miR-126-encoding nucleic acid segment under the control of a promoter active in a target cell, such as a cardiac cell, a smooth muscle cell, an endothelial cell or a hematopoietic cell, a bone marrow cell or an epicardial cell. The promoter may be a tissue selective/specific promoter, with a tissue selective/specific promoter being one active in a cardiac cell, a smooth muscle cell, an endothelial cell, a hematopoietic cell, a bone marrow cell or an epicardial cell. The expression vector may be a viral vector or a non-viral vector.

Administering may comprise systemic administration, such as by oral, intravenous, or intra-arterial routes. Administering may also be by osmotic pump or catheter. Administration can be made directly to or local to vascular damaged tissue or a tissue at risk of vascular damage, such as to cardiac tissue, blood vessel tissue, bone tissue, neuronal tissue, respiratory tissue, eye tissue or placental tissue. The agonist may be contacted with the patient, tissue or site more than once, such as 2, 3, 4, 5, 6, 7, 8, 9, 10, 15, 20, 25, 30, 35, 40, 45, 50, 60, 70, 80, 90 or 100 times. The agonist may be contacted with said tissue over 2, 3, 4, 5, or 6 days, 1, 2, 3, or 4 weeks, 1, 2, 3, 4, 5, 6, 7, 8, 9, 10 or 11 months, or 1, 2, 3, 3, 4, 5, 6, 7, 8, 9, 10, 15, 20, or 25 years.

In another embodiment, there is provided a method of inhibiting pathologic vascularization comprising contacting a subject at risk of or suffering from pathologic vascularization an antagonist of miR-126. The subject suffering from pathologic vascularization may be affected by atherosclerosis, retinopathy, cancer or stroke. The subject at risk of pathologic vascularization may suffer from atherosclerosis, obesity, asthma, arthritis, psoriasis and/or blindness. The subject may be a non-human animal or a human. The antagonist may be a miR-126 antagomir. The target tissue may be vasculature, smooth muscle, ocular tissue, hematopoietic tissue, bone marrow, lung tissue or an epicardial tissue. The method may further comprise administering to said subject a secondary anti-angiogenic therapy.

Administering comprises systemic administration, such as by oral, intravenous, intra-arterial routes. Administration may also be directly to or local to pathologic vascularization or a tissue at risk of pathologic vascularization, such as into ocular tissue, a vascular tissue, bone tissue, fat tissue or lung tissue. Administering also may be by osmotic pump or catheter. The antagonist may be contacted with the patient, tissue or site more than once, such as 2, 3, 4, 5, 6, 7, 8, 9, 10, 15, 20, 25, 30, 35, 40, 45, 50, 60, 70, 80, 90 or 100 times. The antagonist may be contacted with said tissue over 2, 3, 4, 5, or 6 days, 1, 2, 3, or 4 weeks, 1, 2, 3, 4, 5, 6, 7, 8, 9, 10 or 11 months, or 1, 2, 3, 3, 4, 5, 6, 7, 8, 9, 10, 15, 20, or 25 years.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The invention may be better understood by reference to one or more of these drawings in combination with the detailed description of specific embodiments presented herein.

FIGS. 1A-B. Endothelial Cell-Specific Expression and Gene Structure of miR-126. (FIG. 1A) Expression of miR-126 in different tissues and cell lines, as detected by northern blot. U6 or 5S rRNA serves as a loading control. Arrows indicate the position of pre-miR-126, and arrowheads indicate the mature miR-126. SMC, mouse smooth muscle cell line; HUVEC, human umbilical vein endothelial cell (EC) line; P19CL6, derivative of P19 embryonic carcinoma cells; C2C12: mouse myoblast cell line; MS1, mouse primary islet ECs transformed with SV40 large T antigen; HAEC, human aortic ECs; SVEC, SV40-transformed mouse EC line; EOMA, mouse hemangioendothelioma-derived line. (FIG. 1B) (SEQ ID NO:1) Structure of the mouse Egfl7 gene. miR-126 (miR-126-3p) and miR-126* (miR-126-5p) are generated as a stem loop encoded by intron 7. Evolutionary conservation of miR-126 is shown (SEQ ID NO:2).

FIGS. 2A-C. Cis-Regulatory Sequences That Direct Endothelial-Specific Expression of Egfl7/miR-126. (FIG. 2A) E12.5 transgenic mouse embryo harboring a lacZ transgene controlled by 5.4 kb 50 flanking DNA upstream of the Egfl7/miR-126 gene. EC specific lacZ expression showing (a) whole mount embryo, and sagittal section in (b) perichondral region, (c) dermis, (d) brain, and (e) outflow tract. Scale bar=50 mm. (FIG. 2B) Schematic diagrams of genomic regions upstream of the Egfl7/miR-126 gene tested for regulation of lacZ in transgenic mice at E12.5. The fraction of transgenic embryos showing endothelial-specific expression of lacZ is shown. Evolutionary conservation of the Egfl7/miR-126 50 flanking region is shown below. The conserved ETS binding sites are highlighted in pink and light blue. (SEQ ID NOS:3-8) (FIG. 2C) Genomic fragment for regulatory region 1 (Region1-Luc) was tested for activation by increasing amounts of an expression plasmid encoding Ets1 or Ets1 mutant lacking the DNA binding domain (Ets1mut) in COS-7 cells. A deletion mutation was introduced into the ETS binding site (Region1(mut)-Luc). Ets1 activated Region1-Luc but not Region1(mut)-Luc, while Ets1mut failed to activate either construct. Error bars indicate standard deviation.

FIGS. 3A-G. Targeting of the miR-126 Gene. (FIG. 3A) Strategy to generate miR-126 mutant mice by homologous recombination. The 96 bp Egfl7 intron 7 sequence, which contains miR-126, was replaced with a neomycin resistance cassette (Neo) flanked by loxP sites. Neo was removed in the mouse germ line by crossing heterozygous mice to CAG-Cre transgenic mice. DTA, diphtheria toxin A. (FIG. 3B) Southern blot analysis of genomic DNA from ES cells. DNA was digested with Sca I. Using either the 50 probe or 30 probe, the sizes of the wild-type and mutant (miR-126^{neo} allele) are 11.4 kb and 13.4 kb, respectively. Genotypes are shown on the top. (FIG. 3C) Analysis of Egfl7 transcripts in heart (left) or lung (right) of miR-126^{neo/neo} or miR126^{-/-} mice, as detected by RT-PCR. The Egfl7 gene structure and the exon numbers are shown on the bottom. Primers used for RT-PCR were named based on the exon number in the forward (F) and reverse (R) direction. Genotypes are shown on the top. GAPDH was used as a control. Note that Egfl7 expression is disrupted in the miR-126^{neo/neo} mutants, as shown by RT-PCR with primers 7F and 8R, 6F and 9R, 8F and 10R, and normalized upon deletion of the neo cassette, as shown by RT-PCR with the primers indicated. (FIG. 3D) Detection of EGFL7 and GAPDH protein by western blot of heart extracts from WT and miR-126 KO mice. (FIG. 3E) Detection of miR-126 transcripts by northern analysis of hearts and lungs. 5S rRNA serves as a loading control. (FIG. 3F) Genotypes of offspring from miR-126^{+/-} intercrosses. The actual and expected number of mice for each genotype at the indicated stages is shown. (FIG. 3G) Genotypes of embryos from miR-126^{+/-} intercrosses. The number of miR-126^{-/-} offspring analyzed at each age is shown. Severe vascular defects were defined as edema, hemorrhage, severe growth retardation, and lethality. Less than 1% of wild-type or miR-126^{+/-} embryos or neonates showed vascular abnormalities.

FIGS. 4A-F. Vascular Abnormalities in miR-126 Null Mice. (FIG. 4A) Wild-type (WT) and miR-126^{-/-} (KO) embryos at E15.5. A subset of KO embryos shows systemic edema and hemorrhages as indicated by the arrows. (FIG. 4B) Lateral views of cranial regions of WT and miR-126 KO embryos at E10.5. Superficial cranial vessels, shown by arrowheads, are apparent in WT embryos, but are severely deficient in the mutant. The number of vessels in the cranial region indicated by boxes is shown on the right (n=6). (FIG. 4C) Vascularization of the retina at P2 as visualized by PECAM staining. The position of the central retinal artery is demarcated by dashed white lines and the termini of retinal vessels in the mutant by red arrows. Bar=200 mm. Relative vascular coverage is shown on the right (n=3). (FIG. 4D) H&E staining of sagittal sections of the dermis and liver of E15.5 embryos and lung of neonates of the indicated genotypes. Scale bar in the upper and middle panel equals 200 mm, and scale bar in the bottom panel equals 500 mm. The bracket indicates the thickening of dermis with erythrocytes and inflammatory cells in the tissue space in the KO embryo. The arrowheads indicate congestion of red blood cells in KO liver compared to the WT liver. The arrows point to the lungs, in which the alveoli fail to inflate in the KO mice. Asterisks show edema in the thoracic cavity in KO neonates. (FIG. 4E) Electron microscopy of capillaries in WT and KO embryos at E15.5. The bracket shows the breakdown of vessels in KO embryos. The green arrows point to tight junctions in WT endothelial cells. The red arrow points to the red blood cells floating outside of the vessels in KO embryos, while the arrow head indicates the thinning of the endothelial layer in the vessel of KO embryo. EC, endothelial cell; rbc, red blood cell. (FIG. 4F) Endothelial cell proliferation in E15.5 KO embryos. Significantly less BrdU (red) and PECAM1 (green) double-positive cells were observed in KO compared to WT embryos. The red arrow points to the PECAM/BrdU double positive cells, while the white arrow points to the PECAM single-positive cells. Nuclei were stained with DAPI (blue). Error bars indicate standard deviation. The statistics are shown on the bar graph (p=0.014).

FIGS. 5A-E. Impaired Angiogenesis of miR-126 KO EC's. (FIG. 5A) Representative images of cultured aortic rings isolated from wild-type (WT) and miR-126^{-/-} (KO) mice at days 4-6 are shown. Extensive endothelial outgrowth can be seen in WT explants, but not in mutants. Relative migratory activity under each condition was quantified as shown in the bar graphs with statistics. Error bars indicate standard deviation. (FIG. 5B) Representative images of PECAM1 (green) staining of matrigel plugs implanted into mice of the indicated genotypes are shown. Significant less angiogenesis was observed in the matrigel with FGF-2 in KO mice compared to WT mice. No significant angiogenesis was observed in the matrigel plugs with lacking FGF-2 in WT or KO mice. Scale bar=60 mm. (FIG. 5C) The extent of angiogenesis in the matrigel plug assay was quantified by determining PECAM staining area using Image J software. Error bars indicate standard deviation. p=0.0008 for KO compared to WT matrigel plugs. (FIG. 5D) Survival of WT and KO mice following MI. P value equals 0.05 and 0.014 for the survival of KO mice compared to WT for 1 week and 3 week post-MI. (FIG. 5E) Histological analysis of hearts from WT and KO mice following MI. Panels a-d show longitudinal sections through the right ventricle (RV) and left ventricle (LV). Note thrombi in the atria of the mutant heart, indicative of heart failure. Panels e-f show transverse sections stained with Masson's trichrome to reveal scar formation. Note the extensive loss of myocardium in the KO mice. Panels g and h show PECAM1 staining in the boxed infarct region from e and f. Note the deficiency of vasculature in KO mice following MI. The scale bar in panels a-f equals 1 cm, and the scale bar in panels g and h equals 40 mm.

FIGS. 6A-I. Modulation of Angiogenic Growth Factor Signaling by miR-126. (FIG. 6A) miR-126 enhances FGF-dependent phosphorylation of ERK1/2. HUVEC cells were infected with adenovirus expressing lacZ or miR-126, and treated with FGF-2 (10 ng/ml) for the indicated periods of time. Cell lysates were immunoblotted with the indicated antibodies to determine the level of phosphorylated and total ERK1/2. Ad-miR-126 enhanced FGF-2 dependent phosphorylation of ERK1/2. (FIG. 6B) Knockdown of miR-126 diminishes VEGF-dependent phosphorylation of ERK1/2. HAEC cells were transfected with 2′-O-methyl-miR-126 antisense oligonucleotide or control oligonucleotide, and treated with VEGF (10 ng/ml) for 10 min. Cell lysates were immunoblotted with the indicated antibodies to determine the level of phosphorylated and total ERK1/2. GAPDH was used as a loading control. (FIG. 6C) Sequence alignment of miR-126 with Spred-1 30 untranslated regions (UTRs) from different species (SEQ ID NOS:9-14). (FIG. 6D) Detection of Spred-1, CRK, and GAPDH protein by western blot of yolk sac extracts of E15.5 WT and miR-126 KO embryos. (FIG. 6E) miR-126 targets the Spred-1 30 UTR. The 3′ UTR of Spred-1 mRNA, and the Spred-1 m 3′ UTR with mutations engineered in the region complementary to the miR-126 seed region (GGTACGA to TTGGAAG), was inserted into the pMIR-REPORT vector (Ambion). The miR-126 mutant (miR-126 m) construct consists of the miR-126-3p sequence CGTACC mutated to GCATGG, and the corresponding miR-126-5p sequence GGTACG mutated to CCATGC. Transfection of COS-7 cells was performed using the indicated combination of plasmids. CMV-bGAL was used as an internal control for transfection efficiency. Error bars indicate standard deviation. P values are shown; ns, not significant. (FIG. 6F) Relative Spred-1 mRNA expression level upon miR-126 overexpression or knockdown. HUVEC or HAEC cells were subjected to the indicated treatments, and the level of Spred-1 mRNA was determined by real-time PCR. GAPDH served as a control. Error bars indicate standard deviation. P values are shown. (FIG. 6G) Upregulation of Spred-1 mRNA in miR-126^{-/-} endothelial cells. The level of Spred-1 mRNA was determined by real-time PCR with L7 as control. Error bars indicate standard deviation. p=0.01 for KO compared to WT. (FIG. 6H) Representative images of cultured aortic rings isolated from wild-type (WT) and miR-126 KO mice at days 5 and 6 are shown. Adenoviral overexpression of Spred-1 in WT explants impairs endothelial outgrowth, whereas siRNA-mediated knockdown of Spred-1 in explants from miR-126 KO mice enhances endothelial outgrowth. Relative migratory activity under each condition was quantified as shown in the bar graphs with statistics. Error bars indicate standard deviation. (FIG. 6I) Scratch-wound assay of HUVEC cells response to VEGF. Knockdown of miR-126 expression with antisense RNA impairs EC migration, whereas knockdown of Spred-1 with siRNA restores migration in the presence of miR-126 antisense RNA. The edges of the scratch-wound are shown by red dashed lines. Migrated cells were quantified as shown in the bar graphs with statistics. Error bars indicate standard deviation.

FIG. 7. A Model for the Function of miR-126 in Angiogenesis. Binding of VEGF and FGF to their receptors on ECs leads to activation of the MAP kinase signaling pathway, which culminates in the nucleus to stimulate the transcription of genes involved in angiogenesis. miR-126 represses the expression of Spred-1, a negative regulator of Ras/MAP kinase signaling. Thus, loss of miR-126 function diminishes MAP kinase signaling in response to VEGF and FGF, whereas gain of miR-126 function enhances angiogenic signaling.

FIG. 8. Endothelial cell specific expression of pri-miR-126 and its host gene Egfl7. Intron 7 was used as probe for pri-miR-126, while Egfl7 cDNA fragment was used as probe for Egfl7. Labels indicate endothelial cell specific expression of pri-miR-126 and Egfl7 in intersomitic vessel (black arrowhead), dorsal aorta (black arrow), endocardium (white arrowhead) and liver (white arrow) at E8.5, E10.5 and E14.5. Black bar=200 μm, White bar=1600 μm.

### DETAILED DESCRIPTION OF ILLUSTRATIVE EMBODIMENTS

Recent studies have revealed important roles for microRNAs in the response of the cardiovascular system to injury and stress (Latronico et al., 2007; van Rooij and Olson, 2007). miRNAs represent a class of ˜22 nucleotide noncoding RNAs that regulate gene expression by targeting mRNAs for cleavage or translational repression (Bartel, 2004). More than 500 miRNAs have been identified in humans and other eukaryotic species, with about a third encoded by introns of protein coding genes. miRNAs are initially transcribed as large pri-miRNAs that are processed through sequential steps to give rise to a heteroduplex RNA. The miRNA strand of the heteroduplex becomes incorporated into the RNA-induced silencing complex (RISC), where it is enabled to target specific mRNAs through complementary sequences in 3′ untranslated regions (He and Hannon, 2004). The opposite strand of the heteroduplex, known as the star (*) strand, is generally degraded.

The inventors now show that an endothelial cell-specific miRNA, miR-126, modulates angiogenesis in vivo. Targeted deletion of miR-126 in mice results in vascular leakage, hemorrhaging, and embryonic lethality in a subset of mutant mice. These vascular abnormalities can be attributed to diminished angiogenic growth factor signaling, resulting in reduced EC growth, sprouting, and adhesion. The subset of mutant animals that survives is prone to cardiac rupture and lethality following myocardial infarction with defective vascularization of the infarct. The pro-angiogenic actions of miR-126 correlate with its repression of Spred-1, a negative regulator of MAP kinase signaling. Thus, in the absence of miR-126, increased expression of Spred-1 diminishes the transmission of intracellular angiogenic signals by VEGF and FGF.

Based on these observations, the inventors suggest that miR-126 functions as an endothelial cell-specific regulator of angiogenic signaling. The endothelium plays myriad roles in cardiovascular homeostasis and remodeling during disease, including the control of vascular tone and permeability, smooth muscle cell growth and proliferation, leukocyte adhesion, coagulation, and thrombosis. miRNAs have been implicated in regulating EC gene expression and function in vitro (Kuehbacher et al., 2007; Suarez et al., 2007), but the functions of miRNAs in EC biology in vivo have not been explored. The discovery that miR-126 is required for vascular integrity and angiogenesis, as well as survival post-MI, suggests that strategies to elevate miR-126 in the ischemic myocardium could enhance cardiac repair. Conversely, diminishing miR-126 expression may be efficacious in settings of pathological vascularization, such as cancer, atherosclerosis, retinopathy, and stroke. Recently, miR-126 was reported to inhibit tumorigenesis and to be downregulated in metastatic breast tumors, although the specific cell type in which it was downregulated and the targets of miR-126 that might mediate these actions were not defined (Tavazoie et al., 2008). The inventors speculate that miR-126 and other miRNAs will be found to play key roles in tissue remodeling and diseases. These and other aspects of the invention are discussed in detail below.

### I. MIRNAS

A. Background

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are transcribed by RNA polymerase II and can be derived from individual miRNA genes, from introns of protein coding genes, or from poly-cistronic transcripts that often encode multiple, closely related miRNAs. Pre-miRNAs, generally several thousand bases long are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression.

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli, 2002). Several hundred miRNAs have been identified in C. elegans, Drosophila, mouse, and humans. As would be expected for molecules that regulate gene expression, miRNA levels have been shown to vary between tissues and developmental states. In addition, one study shows a strong correlation between reduced expression of two miRNAs and chronic lymphocytic leukemia, providing a possible link between miRNAs and cancer (Calin, 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis and fat metabolism. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). miR-14 has also been implicated in fat metabolism (Xu et al., 2003). Lsy-6 and miR-273 are C. elegans miRNAs that regulate asymmetry in chemosensory neurons (Chang et al., 2004). Another animal miRNA that regulates cell differentiation is miR-181, which guides hematopoietic cell differentiation (Chen et al., 2004). These molecules represent the full range of animal miRNAs with known functions. Enhanced understanding of the functions of miRNAs will undoubtedly reveal regulatory networks that contribute to normal development, differentiation, inter- and intracellular communication, cell cycle, angiogenesis, apoptosis, and many other cellular processes. Given their important roles in many biological functions, it is likely that miRNAs will offer important points for therapeutic intervention or diagnostic analysis.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004), and others have proven their functionality in the heart, where they efficiently knocked-down miR-133 and miR-1 (Care et al. 2007; Yang et al. 2007). Another publication describes the use of plasmids that are transcribed by endogenous RNA polymerases and yield specific miRNAs when transfected into cells (Zeng et al., 2002). These two reagent sets have been used to evaluate single miRNAs.

B. miR-126

In light of recent studies implicating miRNAs in cardiovascular development and disease, the inventors searched publicly available databases for miRNAs that appeared to be restricted to cardiovascular tissues. Among several such miRNAs, miR-126 appeared enriched in tissues with a high vascular component, such as heart and lung (Lagos-Quintana et al., 2002). A survey of miRNA expression patterns in zebrafish also showed miR-126 to be specific for the vascular system (Wienholds et al., 2005).

Northern blot analysis showed miR-126 to be expressed in a broad range of tissues, with highest expression in lung and heart, consistent with prior studies (Harris et al., 2008; Lagos-Quintana et al., 2002; Musiyenko et al., 2008). miR-126* was detectable at only trace levels of expression (data not shown). A survey of cell lines revealed miR-126 to be expressed in primary human umbilical vein ECs (HUVECs) and in numerous EC cell lines, including the MS1, HAEC, and EOMA cell lines, but not in SV40 transformed ECs (SVECs) or nonendothelial cell types.

miR-126 (also referred to as miR-126-3p) and miR-126* (miR-126-5p) are conserved from Fugu to Homo sapiens (microrna.sanger.ac.uk/sequences/index.shtml). In mammals and birds, miR-126 and -126* are encoded by intron 7 of the EGF-like domain 7 (Egfl7) gene, which encodes an EC-specific secreted peptide that has been reported to act as a chemoattractant and inhibitor of smooth muscle cell migration (Campagnolo et al., 2005; Fitch et al., 2004; Parker et al., 2004; Soncin et al., 2003). The expression pattern of miR-126 in tissues and cell lines parallels that of Egfl7 (Fitch et al., 2004; Soncin et al., 2003), consistent with the conclusion that the miRNA is processed from intronic RNA sequence of the pre-Egfl7 mRNA.

miR-126's from a variety of different organisms (mouse, human, rat, dog, chicken zebrafish, fugu) have been identified and the sequence is completely conserved:

| | |
|---|---|
| UCGUACCGUGAGUAAUAAUGCG | (SEQ ID NO: 1) |

C. Agonists and Antagonists of miR-126

Agonists of miR-126 will generally take one of three forms. First, there is miR-126 itself. Such molecules may be delivered to target cells, for example, by injection or infusion, optionally in the a delivery vehicle such as a lipid, such as a liposome or lipid emulsion. Second, one may use expression vectors that drive the expression of miR-126. The composition and construction of various expression vectors is described elsewhere in the document. Third, one may use agents distinct from miR-126 that act up-regulate, stabilize or otherwise enhance the activity of miR-126, including small molecules. Such molecules include “mimetics,” molecules which mimic the function, and possibly form of miR-126, but are distinct in chemical structure.

Antagonism of miRNA function may be achieved by “antagomirs.” Initially described by Krützfeldt and colleagues (Krützfeldt et al., 2005), antagomirs are single-stranded, chemically-modified ribonucleotides that are at least partially complementary to the miRNA sequence. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a cholesterol moiety at its 3′ end. Antagomirs suitable for inhibiting miRNAs may be about 15 to about 50 nucleotides in length, more preferably about 18 to about 30 nucleotides in length, and most preferably about 20 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

Inhibition of miRNA function may also be achieved by administering antisense oligonucleotides. The antisense oligonucleotides may be ribonucleotides or deoxyribonucleotides. Preferably, the antisense oligonucleotides have at least one chemical modification. Antisense oligonucleotides may be comprised of one or more “locked nucleic acids.” “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g., 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Particular antisense oligonucleotides useful for inhibiting the activity of microRNAs are about 19 to about 25 nucleotides in length. Antisense oligonucleotides may comprise a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, that is at least about 95%, 96%, 97%, 98%, or 99% complementary to a target polynucleotide sequence. In one embodiment, the antisense oligonucleotide comprises a sequence that is 100% complementary to a mature miRNA sequence.

Another approach for inhibiting the function of miR-126 is administering an inhibitory RNA molecule having at least partial sequence identity to the mature miR-126 sequence. The inhibitory RNA molecule may be a double-stranded, small interfering RNA (siRNA) or a short hairpin RNA molecule (shRNA) comprising a stem-loop structure. The double-stranded regions of the inhibitory RNA molecule may comprise a sequence that is at least partially identical, e.g., about 75%, 80%, 85%, 90%, 95%, 96%, 97%, 98%, or 99% identical, to the mature miRNA sequence. In some embodiments, the double-stranded regions of the inhibitory RNA comprise a sequence that is at least substantially identical to the mature miRNA sequence. “Substantially identical” refers to a sequence that is at least about 95%, 96%, 97%, 98%, or 99% identical to a target polynucleotide sequence. In other embodiments, the double-stranded regions of the inhibitory RNA molecule may contain 100% identity to the target miRNA sequence.

In other embodiments of the invention, inhibitors of miR-126 may be inhibitory RNA molecules, such as ribozymes, siRNAs, or shRNAs. In one embodiment, an inhibitor of miR-499 is an inhibitory RNA molecule comprising a double-stranded region, wherein the double-stranded region comprises a sequence having 100% identity to the mature miR-126 sequence. In some embodiments, inhibitors are inhibitory RNA molecules which comprise a double-stranded region, wherein said double-stranded region comprises a sequence of at least about 75%, 80%, 85%, 90%, 95%, 96%, 97%, 98%, or 99% identity to the mature miR-126 sequence.

### II. ANGIOGENESIS, MIR-126 AND SPRED-1

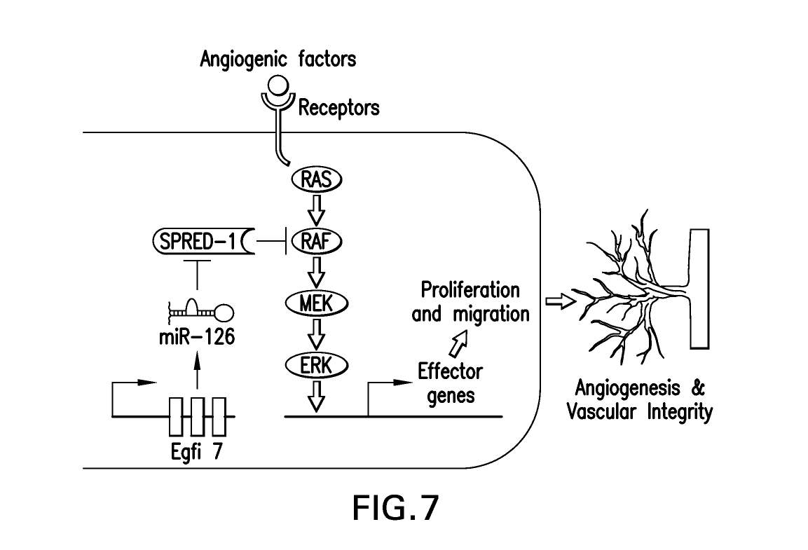

The results of the studies reported below reveal an essential role for miR-126 in angiogenesis and maintenance of vascular integrity in vivo. The actions of miR-126 appear to reflect, at least in part, its potentiation of MAP kinase signaling downstream of VEGF and FGF, which act as potent inducers of angiogenesis (FIG. 7). Spred-1, an intracellular inhibitor of the Ras/MAP kinase pathway, serves as a target for repression by miR-126. Thus, in the absence of miR-126, Spred-1 expression is elevated, resulting in repression of angiogenic signaling. Conversely, miR-126 overexpression relieves the repressive influence of Spred-1 on the signaling pathways activated by VEGF and FGF, favoring angiogenesis.

Consistent with these findings, overexpression of Spred-1 in ECs impairs angiogenesis and cell migration, mimicking the miR-126 loss-of-function phenotype, whereas knockdown of Spred-1 expression enhances angiogenesis and rescues the miR-126 loss-of-function phenotype in cultured ECs. It is intriguing that only a subset of miR-126^{-/-} embryos succumbs to embryonic lethality from vascular rupture, whereas others survive to adulthood, suggesting that this miRNA acts to modulate gene expression programs, rather than functioning as an “on-off” switch for angiogenesis.

In addition to its requirement in normal vascular development during embryogenesis, the functions of miR-126 appear to be important following MI, when injured vessels at the site of the infarct initiate neoangiogenesis to restore blood flow to the injured myocardial wall. Under conditions of stress, as in the heart following MI, the actions of miR-126 may acquire heightened importance due to the requirement of angiogenic signaling for neovascularization. In this regard, VEGF and FGF expression increases in response to myocardial ischemia and is critical for the development of collateral vessels in the ischemic myocardium (Semenza, 2003). Cardiac injury, in addition to activating the migration and proliferation of nearby ECs, results in the homing of circulating hematopoietic progenitor cells to sites of ischemia and their contribution to cardiac repair (Kocher et al., 2001; Takahashi et al., 1999). miR-126 is expressed in hematopoietic stem cells, and might therefore contribute to the regenerative functions of this cell population (Garzon et al., 2006; Landgraf et al., 2007).

A. Control of Angiogenesis by miR-126

Angiogenic growth factors, such as VEGF and FGF, modulate EC proliferation, migration, and adhesion by activating the MAP kinase pathway, which culminates in the nucleus to enhance the expression of genes required for angiogenesis and vascular integrity. The abnormalities associated with miR-126 loss-of-function are similar to the vascular defects resulting from the inhibition of MAP kinase signaling in ECs (Hayashi et al., 2004).

Consistent with the conclusion that miR-126 promotes angiogenesis by dampening the expression of Spred-1, Spred-1 inhibits cell motility and Rho-mediated actin reorganization (Miyoshi et al., 2004), processes important for angiogenesis. Spred-1, and other members of the Spred family, function as membrane-associated suppressors of growth factor-induced ERK activation and block cell proliferation and migration in response to growth factor signaling (Wakioka et al., 2001). The inhibitory actions of Spred proteins are mediated by interference of phosphorylation and activation of Raf, an upstream activator of the MAP kinase pathway. Among the three Spred proteins, only Spred-1 contains a predicted target sequence for miR-126. While these results are consistent with the conclusion that Spred-1 plays a major role as a mediator of the proangiogenic actions of miR-126, it is likely that the actions of miR-126 reflect the combined functions of multiple target proteins that modulate angiogenesis and vascular integrity.

Thus, the present invention provides a method of promoting angiogenesis in a subject in need thereof comprising administering to the subject at least one agonist of miR-126. In one embodiment, the method further comprises administering a second pro-angiogenesis agent. The second pro-angiogenesis may include, but is not limited to, VEGF, FGF, IL-8, CCN1 and Ang-2.

In another embodiment, the present invention provides a method of modulating MAP kinase signaling in a cell comprising contacting the cell with at least one modulator of miR-126 activity. In some embodiments, MAP kinase signaling is reduced in a cell contacted with a miR-126 antagonist. In other embodiments, MAP kinase signaling is enhanced in a cell contacted with a miR-126 agonist. In one embodiment, the cell is an endothelial cell. In another embodiment, the cell is a hematopoietic stem cell.

B. Biogenesis of miR-126

Based on the coexpression of miR-126 and Egfl7 mRNA, as well as the inventors' finding that miR-126 is generated from a retained intron in a subset of Egfl7 pre-mRNAs (unpublished data), the inventors conclude that miR-126 originates from the Egfl7 pre-mRNA. While there are intronic miRNAs that are transcribed independently of their host genes, in all cases to date these miRNAs are transcribed on the opposite strand of the mRNA, in contrast to miR-126 and Egfl7 mRNA. Moreover, Ets binding sites are required for endothelial-specific expression and no such sites are present in intron 7 of the Egfl7 gene.

Recently, Egfl7 knockout mice were reported to display vascular abnormalities remarkably similar to those of miR-126-null mice (Schmidt et al., 2007). The deletion mutation in those mice was reported to result in the absence of an Egfl7 transcript, suggesting that miR-126 expression is also eliminated. However, miR-126 expression was not examined. Thus, the possibility that the phenotype of those mutant mice actually reflects the loss-of-function of miR-126 warrants consideration.

It is becoming increasingly apparent that the integration of miRNAs into introns of protein coding genes represents a common mechanism for coordinating the expression and regulatory functions of miRNAs with protein coding genes. As another example of this form of coregulation, the inventors showed previously that miR-208, which is encoded by an intron of the α-myosin heavy chain (MHC) gene, functions within a regulatory network to control cardiac stress response (van Rooij et al., 2007). Incorporation of a miRNA into an intron of a tissue-specific gene provides an efficient mechanism for ensuring the coregulation of the miRNA with the gene programs it regulates.

### III. METHODS OF TREATING DISEASE STATES

The present invention provides methods of treating various disease states by administering to a subject agonists or antagonists of miR-126. For the purposes of the present application, treatment comprises reducing one or more of the symptoms of associated with the disease states discussed below. Any level of improvement will be considered treatment, and there is no requirement for a particular level of improvement or a “cure.” It is also sufficient in treatment that symptoms be stabilized, i.e., that the disease condition will not worsen.

The present invention provides a method of promoting vascular integrity and/or vascular repair comprising administering to a subject suffering from a vascular condition an agonist of miR-126 function. The vascular condition may include, but is not limited to, myocardial infarction, ischemia-reperfusion injury, stenosis, fibrosis, vascular trauma, and vascular leakage.

A. Conditions Impairing Vascular Integrity and/or Causing the Need for Vascular Repair

As discussed above, the present invention provides for the use of agonists of miR-126 to improve the integrity of vascular tissue, and also to promote vascular repair and neovascularization following injury, including ischemic insults. The following disease states/conditions are specifically contemplated for treatment according to the present invention, but are not limiting.

Treatment regimens would vary depending on the clinical situation. However, long-term maintenance would appear to be appropriate in most circumstances. It also may be desirable treat vascular conditions with modulators of miR-126 intermittently, such as within a brief window during disease progression.

Myocardial infarction. Myocardial infarction (MI), occurs when the blood supply to part of the heart is interrupted. This is most commonly due to occlusion (blockage) of a coronary artery following the rupture of a vulnerable atherosclerotic plaque, which is an unstable collection of lipids (like cholesterol) and white blood cells (especially macrophages) in the wall of an artery. The resulting ischemia (restriction in blood supply) and oxygen shortage, if left untreated for a sufficient period, can cause damage and/or death (infarction) of heart muscle tissue (myocardium).

Classical symptoms of acute myocardial infarction (AMI) include sudden chest pain (typically radiating to the left arm or left side of the neck), shortness of breath, nausea, vomiting, palpitations, sweating, and anxiety (often described as a sense of impending doom). Women may experience fewer typical symptoms than men, most commonly shortness of breath, weakness, a feeling of indigestion, and fatigue. Approximately one quarter of all myocardial infarctions are silent, without chest pain or other symptoms. A heart attack is a medical emergency, and people experiencing chest pain are advised to alert their emergency medical services, because prompt treatment is beneficial.

Immediate treatment for suspected acute myocardial infarction includes oxygen, aspirin, and sublingual glyceryl trinitrate (colloquially referred to as nitroglycerin and abbreviated as NTG or GTN). Pain relief is also often given, classically morphine sulfate. The patient will receive a number of diagnostic tests, such as an electrocardiogram (ECG, EKG), a chest X-ray and blood tests to detect elevations in cardiac markers (blood tests to detect heart muscle damage). The most often used markers are the creatine kinase-MB (CK-MB) fraction and the troponin I (TnI) or troponin T (TnT) levels. On the basis of the ECG, a distinction is made between ST elevation MI (STEMI) or non-ST elevation MI (NSTEMI). Most cases of STEMI are treated with thrombolysis or if possible with percutaneous coronary intervention (PCI, angioplasty and stent insertion), provided the hospital has facilities for coronary angiography. NSTEMI is managed with medication, although PCI is often performed during hospital admission. In patients who have multiple blockages and who are relatively stable, or in a few extraordinary emergency cases, bypass surgery of the blocked coronary artery is an option.

Ischemia-reperfusion injury. Ischemia-reperfusion injury is caused at least in part by the inflammatory response of damaged tissues. White blood cells carried to the area by the newly returning blood release a host of inflammatory factors such as interleukins as well as free radicals in response to tissue damage. The restored blood flow reintroduces oxygen within cells that damages cellular proteins, DNA and the plasma membrane. Damage to the cell's membrane may in turn cause the release of more free radicals. Such reactive species may also act indirectly in redox signaling to turn on apoptosis. Leukocytes may also build up in small capillaries, obstructing them and leading to more ischemia.

Reperfusion injury plays a part in the brain's ischemic cascade, which is involved in stroke and brain trauma. Repeated bouts of ischemia and reperfusion injury also are thought to be a factor leading to the formation and failure to heal of chronic wounds such as pressure sores and diabetic foot ulcers. Continuous pressure limits blood supply and causes ischemia, and the inflammation occurs during reperfusion. As this process is repeated, it eventually damages tissue enough to cause a wound.

Glisodin, a dietary supplement derived from superoxide dismutase (SOD) and wheat gliadin, has been studied for its ability to mitigate ischemia-reperfusion injury. A study of aortic cross-clamping, a common procedure in cardiac surgery, demonstrated a strong potential benefit with further research ongoing.

Stenosis. A stenosis is an abnormal narrowing in a blood vessel or other tubular organ or structure. Stenoses of the vascular type are often associated with a noise (bruit) resulting from turbulent flow over the narrowed blood vessel. This bruit can be made audible by a stethoscope. Other, more reliable methods of diagnosing a stenosis are imaging methods including ultrasound, Magnetic Resonance Imaging/Magnetic Resonance Angiography, Computed Tomography/CT-Angiography which combine anatomic imaging (i.e., the visible narrowing of a vessel) with the display of flow phenomena (visualization of the movement of the bodily fluid through the bodily structure). Vascular stenoses include intermittent claudication (peripheral artery stenosis), angina (coronary artery stenosis), carotid artery stenosis which predispose to (strokes and transient ischaemic episodes) and renal artery stenosis.

Other Conditions. Trauma and vascular leakage are also conditions which may be treated with miR-126 or agonists thereof.

Risks. The present invention also contemplates treating individuals at risk for any of the aforementioned disease states. These individuals would include those persons suffering from fibrosis. hypertension, cardiac hypertrophy, osteoporosis, neurodegeneration, and/or respiratory distress.

B. Pathologic Neovascularization

As discussed above, the present invention provides for the use of antagonists of miR-126 to impede neovasculariziation that leads to or contributes to disease. The following disease states/conditions are specifically contemplated for treatment accordin to the present invention, but are not limiting.

The present invention provides a method of inhibiting pathologic vascularization in a subject in need thereof comprising administering to a subject an antagonist of miR-126. A condition associated with pathologic vascularization includes, but is not limited to, atherosclerosis, retinopathy, cancer, and stroke.

Early Stage Atherosclerosis. Atherosclerosis is a disease affecting arterial blood vessels. It is a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL). It is commonly referred to as a “hardening” of the arteries. It is caused by the formation of multiple plaques within the arteries.

Atherosclerosis develops from low-density lipoprotein cholesterol (LDL), colloquially called “bad cholesterol.” When this lipoprotein gets through the wall of an artery, oxygen free radicals react with it to form oxidized-LDL. The body's immune system responds by sending specialised white blood cells (macrophages and T-lymphocytes) to absorb the oxidized-LDL. Unfortunately, these white blood cells are not able to process the oxidized-LDL, and ultimately grow then rupture, depositing a greater amount of oxidized cholesterol into the artery wall. This triggers more white blood cells, continuing the cycle. Eventually, the artery becomes inflamed. The cholesterol plaque causes the muscle cells to enlarge and form a hard cover over the affected area. This hard cover is what causes a narrowing of the artery, reduces the blood flow and increases blood pressure.

Atherosclerosis typically begins in early adolescence, and is usually found in most major arteries, yet is asymptomatic and not detected by most diagnostic methods during life. The stage immediately prior to actual atherosclerosis is known as subclinical atherosclerosis. It most commonly becomes seriously symptomatic when interfering with the coronary circulation supplying the heart or cerebral circulation supplying the brain, and is considered the most important underlying cause of strokes, heart attacks, various heart diseases including congestive heart failure, and most cardiovascular diseases, in general. Atheroma in arm, or more often in leg arteries, which produces decreased blood flow is called Peripheral artery occlusive disease (PAOD). Most artery flow disrupting events occur at locations with less than 50% lumen narrowing (˜20% stenosis is average).

Although the disease process tends to be slowly progressive over decades, it usually remains asymptomatic until an atheroma obstructs the bloodstream in the artery. This is typically by rupture of an atheroma, clotting and fibrous organization of the clot within the lumen, covering the rupture but also producing stenosis, or over time and after repeated ruptures, resulting in a persistent, usually localized stenosis. Stenoses can be slowly progressive, whereas plaque rupture is a sudden event that occurs specifically in atheromas with thinner/weaker fibrous caps that have become “unstable.”

Repeated plaque ruptures, ones not resulting in total lumen closure, combined with the clot patch over the rupture and healing response to stabilize the clot, is the process that produces most stenoses over time. The stenotic areas tend to become more stable, despite increased flow velocities at these narrowings. Most major blood-flow-stopping events occur at large plaques, which, prior to their rupture, produced very little if any stenosis.

From clinical trials, 20% is the average stenosis at plaques that subsequently rupture with resulting complete artery closure. Most severe clinical events do not occur at plaques that produce high-grade stenosis. From clinical trials, only 14% of heart attacks occur from artery closure at plaques producing a 75% or greater stenosis prior to the vessel closing.

If the fibrous cap separating a soft atheroma from the bloodstream within the artery ruptures, tissue fragments are exposed and released, and blood enters the atheroma within the wall and sometimes results in a sudden expansion of the atheroma size. Tissue fragments are very clot-promoting, containing collagen and tissue factor; they activate platelets and activate the system of coagulation. The result is the formation of a thrombus (blood clot) overlying the atheroma, which obstructs blood flow acutely. With the obstruction of blood flow, downstream tissues are starved of oxygen and nutrients. If this is the myocardium (heart muscle), angina (cardiac chest pain) or myocardial infarction (heart attack) develops.

If atherosclerosis leads to symptoms, some symptoms such as angina pectoris can be treated. Non-pharmaceutical means are usually the first method of treatment, such as cessation of smoking and practicing regular exercise. If these methods do not work, medicines are usually the next step in treating cardiovascular diseases, and, with improvements, have increasingly become the most effective method over the long term. However, medicines are criticized for their expense, patented control and occasional undesired effects.

In general, the group of medications referred to as statins has been the most popular and are widely prescribed for treating atherosclerosis. They have relatively few short-term or longer-term undesirable side-effects, and multiple comparative treatment/placebo trials have fairly consistently shown strong effects in reducing atherosclerotic disease ‘events’ and generally ˜25% comparative mortality reduction in clinical trials, although one study design, ALLHAT, was less strongly favorable.

The newest statin, rosuvastatin, has been the first to demonstrate regression of atherosclerotic plaque within the coronary arteries by IVUS (intravascular ultrasound evaluation), The study was set up to demonstrate effect primarily on atherosclerosis volume within a 2 year time-frame in people with active/symptomatic disease (angina frequency also declined markedly) but not global clinical outcomes, which was expected to require longer trial time periods; these longer trials remain in progress.

However, for most people, changing their physiologic behaviors, from the usual high risk to greatly reduced risk, requires a combination of several compounds, taken on a daily basis and indefinitely. More and more human treatment trials have been done and are ongoing that demonstrate improved outcome for those people using more-complex and effective treatment regimens that change physiologic behaviour patterns to more closely resemble those that humans exhibit in childhood at a time before fatty streaks begin forming.

Retinopathy. Retinopathy is a general term that refers to some form of non-inflammatory damage to the retina of the eye. Most commonly it is a problem with the blood supply that is the cause for this condition. Frequently, retinopathy is an ocular manifestation of systemic disease. Retinopathy is diagnosed by an optometrist or an ophthalmologist during ophthalmoscopy. Treatment depends on the cause of the disease.

The main causes of retinopathy are diabetes—diabetic retinopathy; arterial hypertension—hypertensive retinopathy; prematurity of the newborn—retinopathy of prematurity (ROP); sickle cell anemia; genetic retinopathy; direct sunlight exposure—solar retinopathy; medicinal products—drug-related retinopathy; and retinal vein or artery occlusion. Many types of retinopathy are progressive and may result in blindness or severe vision loss or impairment, particularly if the macula becomes affected.

Stroke. Stroke is the rapidly developing loss of brain functions due to a disturbance in the blood vessels supplying blood to the brain. This can be due to ischemia (lack of blood supply) caused by thrombosis or embolism, or due to a hemorrhage. It can cause permanent neurological damage, complications and death if not promptly diagnosed and treated. Risk factors for stroke include advanced age, hypertension (high blood pressure), previous stroke or transient ischemic attack (TIA), diabetes, high cholesterol, cigarette smoking, atrial fibrillation, estrogen-containing forms of hormonal contraception, migraine with aura, and thrombophilia (a tendency to thrombosis), patent foramen ovale and several rarer disorders. High blood pressure is the most important modifiable risk factor of stroke.

The traditional definition of stroke, devised by the World Health Organization in the 1970s, is a “neurological deficit of cerebrovascular cause that persists beyond 24 hours or is interrupted by death within 24 hours.” The 24-hour limit divides stroke from transient ischemic attack, which is a related syndrome of stroke symptoms that resolve completely within 24 hours. With the availability of treatments that, when given early, can reduce stroke severity, many now prefer alternative concepts, such as brain attack and acute ischemic cerebrovascular syndrome (modeled after heart attack and acute coronary syndrome respectively), that reflect the urgency of stroke symptoms and the need to act swiftly.

Stroke is occasionally treated with thrombolysis (“clot-buster”), but usually with supportive care (physiotherapy and occupational therapy) and secondary prevention with antiplatelet drugs (aspirin and often dipyridamole), blood pressure control, statins and anticoagulation (in selected patients).

Strokes can be classified into two major categories: ischemic and hemorrhagic. Ischemia is due to interruption of the blood supply, while hemorrhage is due to rupture of a blood vessel or an abnormal vascular structure. 80% of strokes are due to ischemia; the remainder are due to hemorrhage.

In an ischemic stroke, blood supply to part of the brain is decreased, leading to dysfunction and necrosis of the brain tissue in that area. There are four reasons why this might happen: thrombosis (obstruction of a blood vessel by a blood clot forming locally), embolism (idem due to an embolus from elsewhere in the body, see below), systemic hypoperfusion (general decrease in blood supply, e.g., in shock) and venous thrombosis. Stroke without an obvious explanation is termed “cryptogenic” (of unknown origin).

In thrombotic stroke, a thrombus (blood clot) usually forms around atherosclerotic plaques. Since blockage of the artery is gradual, onset of symptomatic thrombotic strokes is slower. A thrombus itself (even if non-occluding) can lead to an embolic stroke (see below) if the thrombus breaks off, at which point it is called an “embolus.” Thrombotic stroke can be divided into two types depending on the type of vessel the thrombus is formed on—large vessel disease or small vessel disease.

Embolic stroke refers to the blockage of an artery by an embolus, a traveling particle or debris in the arterial bloodstream originating from elsewhere. An embolus is most frequently a thrombus, but it can also be a number of other substances including fat (e.g. from bone marrow in a broken bone), air, cancer cells or clumps of bacteria (usually from infectious endocarditis). Because an embolus arises from elsewhere, local therapy only solves the problem temporarily. Thus, the source of the embolus must be identified. Because the embolic blockage is sudden in onset, symptoms usually are maximal at start. Also, symptoms may be transient as the embolus is partially resorbed and moves to a different location or dissipates altogether. Emboli most commonly arise from the heart (especially in atrial fibrillation) but may originate from elsewhere in the arterial tree. In paradoxical embolism, a deep vein thrombosis embolises through an atrial or ventricular septal defect in the heart into the brain.

Cardiac causes can be distinguished between high- and low-risk:
High risk: atrial fibrillation and paroxysmal atrial fibrillation, rheumatic disease of the mitral or aortic valve disease, artificial heart valves, known cardiac thrombus of the atrium or vertricle, sick sinus syndrome, sustained atrial flutter, recent myocardial infarction, chronic myocardial infarction together with ejection fraction <28 percent, symptomatic congestive heart failure with ejection fraction <30 percent, dilated cardiomyopathy, Libman-Sacks endocarditis, Marantic endocarditis, infective endocarditis, papillary fibroelastoma, left atrial myxoma and coronary artery bypass graft (CABG) surgery
Low risk/potential: calcification of the annulus (ring) of the mitral valve, patent foramen ovale (PFO), atrial septal aneurysm, atrial septal aneurysm with patent foramen ovale, left ventricular aneurysm without thrombus, isolated left atrial “smoke” on echocardiography (no mitral stenosis or atrial fibrillation), complex atheroma in the ascending aorta or proximal arch

Systemic hypoperfusion is the reduction of blood flow to all parts of the body. It is most commonly due to cardiac pump failure from cardiac arrest or arrhythmias, or from reduced cardiac output as a result of myocardial infarction, pulmonary embolism, pericardial effusion, or bleeding. Hypoxemia (low blood oxygen content) may precipitate the hypoperfusion. Because the reduction in blood flow is global, all parts of the brain may be affected, especially “watershed” areas—border zone regions supplied by the major cerebral arteries. Blood flow to these areas does not necessarily stop, but instead it may lessen to the point where brain damage can occur. This phenomenon is also referred to as “last meadow” to point to the fact that in irrigation the last meadow receives the least amount of water.

Cerebral venous sinus thrombosis leads to stroke due to locally increased venous pressure, which exceeds the pressure generated by the arteries. Infarcts are more likely to undergo hemorrhagic transformation (leaking of blood into the damaged area) than other types of ischemic stroke.

An ischemic stroke is due to a thrombus (blood clot) occluding a cerebral artery, a patient is given antiplatelet medication (aspirin, clopidogrel, dipyridamole), or anticoagulant medication (warfarin), dependent on the cause, when this type of stroke has been found. Hemorrhagic stroke must be ruled out with medical imaging, since this therapy would be harmful to patients with that type of stroke.

Other immediate strategies to protect the brain during stroke include ensuring that blood sugar is as normal as possible (such as commencement of an insulin sliding scale in known diabetics), and that the stroke patient is receiving adequate oxygen and intravenous fluids. The patient may be positioned so that his or her head is flat on the stretcher, rather than sitting up, since studies have shown that this increases blood flow to the brain. Additional therapies for ischemic stroke include aspirin (50 to 325 mg daily), clopidogrel (75 mg daily), and combined aspirin and dipyridamole extended release (25/200 mg twice daily).

It is common for the blood pressure to be elevated immediately following a stroke. Studies indicated that while high blood pressure causes stroke, it is actually beneficial in the emergency period to allow better blood flow to the brain.

If studies show carotid stenosis, and the patient has residual function in the affected side, carotid endarterectomy (surgical removal of the stenosis) may decrease the risk of recurrence if performed rapidly after stroke.

If the stroke has been the result of cardiac arrhythmia with cardiogenic emboli, treatment of the arrhythmia and anticoagulation with warfarin or high-dose aspirin may decrease the risk of recurrence. Stroke prevention treatment for a common arrhythmia, atrial fibrillation, is determined according to the CHADS/CHADS2 system.

In increasing numbers of primary stroke centers, pharmacologic thrombolysis (“clot busting”) with the drug tissue plasminogen activator (tPA), is used to dissolve the clot and unblock the artery. However, the use of tPA in acute stroke is controversial. Another intervention for acute ischemic stroke is removal of the offending thrombus directly. This is accomplished by inserting a catheter into the femoral artery, directing it into the cerebral circulation, and deploying a corkscrew-like device to ensnare the clot, which is then withdrawn from the body. Anticoagulation can prevent recurrent stroke. Among patients with nonvalvular atrial fibrillation, anticoagulation can reduce stroke by 60% while antiplatelet agents can reduce stroke by 20%. However, a recent meta-analysis suggests harm from anti-coagulation started early after an embolic stroke.

Cancer. Cancers comprise a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, do not invade or metastasize. Most cancers form a tumor but some, like leukemia, do not. The branch of medicine concerned with the study, diagnosis, treatment, and prevention of cancer is oncology.

Nearly all cancers are caused by abnormalities in the genetic material of the transformed cells. These abnormalities may be due to the effects of carcinogens, such as tobacco smoke, radiation, chemicals, or infectious agents. Other cancer-promoting genetic abnormalities may be randomly acquired through errors in DNA replication, or are inherited, and thus present in all cells from birth. The heritability of cancers are usually affected by complex interactions between carcinogens and the host's genome. New aspects of the genetics of cancer pathogenesis, such as DNA methylation, and microRNAs are increasingly recognized as important.

Diagnosis usually requires the histologic examination of a tissue biopsy specimen by a pathologist, although the initial indication of malignancy can be symptoms or radiographic imaging abnormalities. Most cancers can be treated and some cured, depending on the specific type, location, and stage. Once diagnosed, cancer is usually treated with a combination of surgery, chemotherapy and radiotherapy.

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma. Radiation dose to each site depends on a number of factors, including the radiosensitivity of each cancer type and whether there are tissues and organs nearby that may be damaged by radiation. Thus, as with every form of treatment, radiation therapy is not without its side effects.

Chemotherapy is the treatment of cancer with drugs that can destroy cancer cells. In current usage, the term “chemotherapy” usually refers to cytotoxic drugs which affect rapidly dividing cells in general, in contrast with targeted therapy (see below). Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can. Hence, chemotherapy has the potential to harm healthy tissue, especially those tissues that have a high replacement rate (e.g., intestinal lining). These cells usually repair themselves after chemotherapy. Because some drugs work better together than alone, two or more drugs are often given at the same time. This is called “combination chemotherapy,” and indeed, most chemotherapy regimens are given in a combination.

Targeted therapy, which first became available in the late 1990's, has had a significant impact in the treatment of some types of cancer, and is currently a very active research area. This constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors imatinib and gefitinib.

Monoclonal antibody therapy is another strategy in which the therapeutic agent is an antibody which specifically binds to a protein on the surface of the cancer cells. Examples include the anti-HER2/neu antibody trastuzumab (Herceptin) used in breast cancer, and the anti-CD20 antibody rituximab, used in a variety of B-cell malignancies.

Targeted therapy can also involve small peptides as “homing devices” which can bind to cell surface receptors or affected extracellular matrix surrounding the tumor. Radionuclides which are attached to this peptides (e.g., RGDs) eventually kill the cancer cell if the nuclide decays in the vicinity of the cell. Especially oligo- or multimers of these binding motifs are of great interest, since this can lead to enhanced tumor specificity and avidity.

Photodynamic therapy (PDT) is a ternary treatment for cancer involving a photosensitizer, tissue oxygen, and light (often using lasers). PDT can be used as treatment for basal cell carcinoma (BCC) or lung cancer; PDT can also be useful in removing traces of malignant tissue after surgical removal of large tumors.

Cancer immunotherapy refers to a diverse set of therapeutic strategies designed to induce the patient's own immune system to fight the tumor. Contemporary methods for generating an immune response against tumours include intravesical BCG immunotherapy for superficial bladder cancer, and use of interferons and other cytokines to induce an immune response in renal cell carcinoma and melanoma patients. Vaccines to generate specific immune responses are the subject of intensive research for a number of tumours, notably malignant melanoma and renal cell carcinoma. Sipuleucel-T is a vaccine-like strategy in late clinical trials for prostate cancer in which dendritic cells from the patient are loaded with prostatic acid phosphatase peptides to induce a specific immune response against prostate-derived cells.

Allogeneic hematopoietic stem cell transplantation (“bone marrow transplantation” from a genetically non-identical donor) can be considered a form of immunotherapy, since the donor's immune cells will often attack the tumor in a phenomenon known as graft-versus-tumor effect. For this reason, allogeneic HSCT leads to a higher cure rate than autologous transplantation for several cancer types, although the side effects are also more severe.

The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

Angiogenesis inhibitors prevent the extensive growth of blood vessels (angiogenesis) that tumors require to survive. Some, such as bevacizumab, have been approved and are in clinical use. One of the main problems with anti-angiogenesis drugs is that many factors stimulate blood vessel growth, in normal cells and cancer. Anti-angiogenesis drugs only target one factor, so the other factors continue to stimulate blood vessel growth. Other problems include route of administration, maintenance of stability and activity and targeting at the tumor vasculature.

Risk. The present invention also contemplates treating individuals at risk for any of the aforementioned disease states. These individuals would include those persons suffering from atherosclerosis, obesity, asthma, arthritis, psoriasis and/or blindness.

C. Combined Therapy

In another embodiment, it is envisioned to use a modulator of miR-126 in combination with other therapeutic modalities. Thus, in addition to the therapies described above, one may also provide to the patient more “standard” pharmaceutical therapies. Combinations may be achieved by contacting cells, tissues or subjects with a single composition or pharmacological formulation that includes both agents, or by contacting the cell with two distinct compositions or formulations, at the same time, wherein one composition includes the expression construct and the other includes the agent. Alternatively, the therapy using a modulator of miR-126 may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell, tissue or subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a modulator of miR-126, or the other agent will be desired. In this regard, various combinations may be employed. By way of illustration, where the modulator of miR-126 is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B
B/B/B/A A/A/A/B B/A/A/A A/B/A/A A/A/B/A A/B/B/B
B/A/B/B B/B/A/B

Other combinations are likewise contemplated.

D. Pharmacological Therapeutic Agents

Pharmacological therapeutic agents and methods of administration, dosages, etc., are well known to those of skill in the art (see for example, the “Physicians Desk Reference,” Klaassen's “The Pharmacological Basis of Therapeutics,” “Remington's Pharmaceutical Sciences,” and “The Merck Index, Eleventh Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. Also contemplated for combination with an miR-126 modulator are any of the agents/therapies discussed in Sections IIIA-B, above.

E. Regulation of Therapies

The present invention also contemplates methods for scavenging or clearing miR-126 agonists or antagonists following treatment. The method may comprise overexpressing binding sites for the miR-126 antagonists in target tissues. In another embodiment, the present invention provides a method for scavenging or clearing miR-126 following treatment. In one embodiment, the method comprises overexpression of binding site regions for miR-126 in target tissues. The binding site regions preferably contain a sequence of the seed region for miR-126. In some embodiments, the binding site may contain a sequence from the 3′ UTR of one or more targets of miR-126, such as Spred-1. In another embodiment, a miR-126 antagonist may be administered after miR-126 to attenuate or stop the function of the miRNA.

F. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the polypeptides of the present invention generally may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including gels, pastes, powders and slurries. The active ingredient may be added in a therapeutically effective amount to a paste dentifrice that may include water, binders, abrasives, flavoring agents, foaming agents, and humectants.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. KITS

Any of the compositions described herein may be comprised in a kit. In a non-limiting example, an individual miRNA modulator (e.g., miRNA, expression construct, antagomir) is included in a kit. The kit may further include water and hybridization buffer to facilitate hybridization of the two strands of the miRNAs. The kit may also include one or more transfection reagent(s) to facilitate delivery of the miRNA to cells.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used for the manipulation or characterization of miRNA.

### V. SCREENING METHODS

The present invention further comprises methods for identifying modulators of miR-126 that are useful in the prevention or treatment of the diseases discussed above. These assays may comprise random screening of large libraries of candidate substances; alternatively, the assays may be used to focus on particular classes of compounds selected with an eye towards structural attributes that are believed to make them more likely to modulate the expression and/or function of miR-126.

To identify a modulator of miR-126, one generally will determine the function of a miR-126 in the presence and absence of the candidate substance. For example, a method generally comprises:
(a) providing a candidate modulator;
(b) admixing the candidate modulator with a miR-126;
(c) measuring miR-126 activity; and
(d) comparing the activity in step (c) with the activity in the absence of the candidate modulator,
wherein a difference between the measured activities indicates that the candidate modulator is, indeed, a modulator of miR-126.
   Assays also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially modulate angiogenic-regulating aspects of miR-126. One will typically acquire, from various commercial sources, molecular libraries that are believed to meet the basic criteria for useful drugs in an effort to “brute force” the identification of useful compounds. Screening of such libraries, including combinatorially-generated libraries (e.g., antagomir libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small nucleic acids may be synthesized on a solid substrate, such as plastic pins or some other surface. Such molecules can be rapidly screening for their ability to inhibit miR-126.

C. In Cyto Assays

The present invention also contemplates the screening of compounds for their ability to modulate miR-126 activity and expression in cells. Various cell lines, including those derived from endothelial cells and hematopoietic cells, can be utilized for such screening assays, including cells specifically engineered for this purpose.

The present invention also contemplate examining the expression of an miR-126 target gene to determine whether miR-126 activity had been modulated. Thus, any of the gene targets set out in Table 3 or 4 could be examined as part of a screening strategy, and advantageously may look at a plurality of the these targets.

D. In Vivo Assays

In vivo assays involve the use of various animal models of vascular diseases, discussed above, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria. Also, measuring toxicity and dose response can be performed in animals in a more meaningful fashion than in in vitro or in cyto assays.

### VI. VECTORS FOR CLONING, GENE TRANSFER AND EXPRESSION

Within certain embodiments expression vectors are employed to express miR-126 or related molecules (e.g., antagomirs). Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. Generally, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1981; Lee et |
| mammary tumor | | al., 1981; Majors et al., |
| virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid | Thyroid Hormone | Chatterjee et al., 1989 |
| Stimulating | | |
| Hormone α Gene | | |

Of particular interest are endothelial cell promoters, such as Tie1, Tie2, Ve-cadherin, EGFL7/miR-126 or promoters, and muscle specific promoters, including cardiac specific promoters. These include the myosin light chain-2 promoter (Franz et al., 1994; Kelly et al., 1995), the α-actin promoter (Moss et al., 1996), the troponin 1 promoter (Bhaysar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (LaPointe et al., 1996) and the αB-crystallin/small heat shock protein promoter (Gopal-Srivastava, 1995), α-myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubinstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubinstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, the oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### VII. METHODS OF MAKING TRANSGENIC MICE

A particular embodiment of the present invention provides transgenic animals that lack one or both functional miR-126 alleles. Also, transgenic animals that express miR-126 under the control of an inducible, tissue selective or a constitutive promoter, recombinant cell lines derived from such animals, are contemplated. The use of an inducible or repressable miR-126 encoding nucleic acid provides a model for over- or unregulated expression. Also, transgenic animals that are “knocked out” for miR-126, in one or both alleles, are contemplated. Also, transgenic animals that are “knocked out” for miR-126, in one or both alleles for one or both clusters, are contemplated.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; incorporated herein by reference), and Brinster et al. (1985; incorporated herein by reference).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA. Other methods for purification of DNA for microinjection are described in in Palmiter et al. (1982); and in Sambrook et al. (2001).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by C02 asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

### VIII. DEFINITIONS

The term “treatment” or grammatical equivalents encompasses the improvement and/or reversal of the symptoms of disease. “Improvement in the physiologic function” of the heart may be assessed using any of the measurements described herein, as well as any effect upon the animal's survival. In use of animal models, the response of treated transgenic animals and untreated transgenic animals is compared using any of the assays described herein (in addition, treated and untreated non-transgenic animals may be included as controls).

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “agonist” refers to molecules or compounds that mimic or promote the action of a “native” or “natural” compound. Agonists may be homologous to these natural compounds in respect to conformation, charge or other characteristics. Agonists may include proteins, nucleic acids, carbohydrates, small molecule pharmaceuticals or any other molecules that interact with a molecule, receptor, and/or pathway of interest.

As used herein, the terms “antagonist” and “inhibitor” refer to molecules, compounds, or nucleic acids that inhibit the action of a factor. Antagonists may or may not be homologous to these natural compounds in respect to conformation, charge or other characteristics. Antagonists may have allosteric effects that prevent the action of an agonist. Alternatively, antagonists may prevent the function of the agonist. Antagonists and inhibitors may include proteins, nucleic acids, carbohydrates, small molecule pharmaceuticals or any other molecules that bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

### IX. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Generation of miR-126 null mice. To generate the miR-126 targeting vector, a 5.7 kb fragment (5′ arm) extending upstream of the miR-126 coding region and a 1.8 kb fragment (3′ arm) downstream of the miR-126 coding region were cloned into the pGKneoF2L2dta targeting plasmid upstream and downstream of the loxP sites and the Frt-flanked neomycin cassette, respectively. Targeted miR-126 ES-cells were screened by Southern blot analysis, and injected into blastocysts. Germline transmission was obtained from the chimeras and the mutant miR-126 allele was obtained by crossing miR-126^{neo/+} mice with CAG-Cre transgenic mice.

Coronary artery ligation procedure. Male mice, aged from 8-12 weeks old, were subjected to coronary artery ligation for the production of MI by a surgeon blind to the genotype as described (van Rooij et al., 2004). Briefly, the animals were anesthetized with isoflurane, intubated with a 20 G needle, and then ventilated with a volume-cycled rodent respirator with a 0.1 cc/stroke at a respiratory rate of 150 cycles/min. After thoracotomy, ligation of the LAD artery was performed with a 7-0 prolene suture. Sham-operated animals underwent the same procedure without ligation of the left coronary artery. The chest wall was closed with 3 single 5.0 silk sutures, and the skin was closed with topical tissue adehisive (Nexabrand). The animals were then post injected with pain killer buprenorphine HCL, extubated and allowed to recover from surgery under a heating pad (37° C.) for 1 hr. All mice were housed for 1-3 weeks and subject to euthanasia for histology and immunostaining of the hearts. Animal surgical procedures were approved by the UT Southwestern IACUC.

Electron microscopy. Dermis and underlying tissues from the back of wild-type and miR-126^{-/-} E15.5 embryos were dissected and fixed in 2% glutaraldehyde in PBS for 24 hrs. The samples were post-fixed and stained with 1% osmium tetroxide in PBS, stained, then dehydrated in a series of graded ethanol and propylene oxide. The tissues were subsequently embedded in Eponate 12, counterstained with uranyl acetate and lead citrate, and sectioned at 90 nm.

Endothelial cell isolation. EC cells were isolated from the kidney using rat anti-PECAM1 antibody (BD Pharmingen) and Danabeads (Dana Biotech) as described (Marelli-Berg et al., 2000). Briefly, minced kidneys were digested with Collagenae/Dispase mix (3 mg/ml, Roch) and 0.005% DNase at 37° C. for 30 min with agitation. The cells were then filtered through 40 μm nylon mesh, and washed twice with DMEM plus 5% FCS. After preplated for 1 hr on the plates coated with 0.1% gelatin, the floating cells were tranfered to another coated plate and grown overnight. The cells were trypsinized and blocked with murine immunoglobin (Chemicon) for 30 min, and incubated with anti-PECAM1 at 4° C. for another 30 min. After several washes, the cells were incubated with Danabeads sheep anti-rat IgG at 4° C. for 30 min. Unbound cells were washed away with PBS/0.5% FCS, and remaining cells were plated on gelatin-coated plates for further analysis. An aliquot of cells was stained with DiI-Ac-LDL (Biomedical Tech) to evaluate purity.

RNA and Western blot analysis. Total RNA was isolated from mouse tissues or cell lines using TRIzol reagent (Invitrogen). Northern blots to detect microRNAs were performed as described previously (van Rooij et al., 2006). Regular RT-PCR or Real-time RT-PCR with Sybergreen probes was performed using 1 μg of RNA as a template with random hexamer primers to generate cDNA. For cloning of miR-126 splicing variants, Race-Ready cDNA from human placenta (Ambion) was used. Sequences of PCR primers are available upon request. For Western blot analysis, protein lysates were resolved by SDS-PAGE and blotted using standard procedures. Spred-1 antibody was kindly provided by Dr. Kai Schuh. Other antibodies used were as follows: CRK (BD Biosciences), ERK1/2 (Cell signaling), Phospho-ERK1/2 (Cell signaling), and GAPDH (Abcam).

RNA in situ hybridization. RNA in situ hybridization was performed as described (Chang et al., 2006). Sense and antisense Egfl7 intron 7, containing miR-126 sequence, and an Egfl7 exon-specific cDNA were used as probes for in situ hybridization of tissue sections.

miRNA Northern Blot. Generation and Analysis of Transgenic Mice Transgenes were generated by cloning DNA fragments from the Egfl7 50 flanking region into the hsp68 basal promoter upstream of a lacZ reporter gene (Kothary et al., 1989). These reporter constructs were injected into fertilized oocytes from B6C3F mice and implanted into pseudopregnant ICR mice. Embryos were collected and stained for b-galactosidase activity. Transgenic embryos were identified by PCR analysis with lacZ primer pairs.

Histology, BrdU Labeling, TUNEL Assay, and Immunohistochemistry. Histology was performed as described (Chang et al., 2006). For BrdU labeling, animals were injected intraperitoneally with 100 μg BrdU/g 4 hr prior to sacrifice. For whole-mount immunostaining, embryos or P2 retinas were fixed in 4% paraformaldehyde for 2 hr and processed for staining with PECAM using standard procedures. For section immunohistochemistry, embryos or mouse hearts were fixed in 4% paraformadehyde overnight, and processed for cryosection and single or double immunostaining using standard procedures. Apoptosis was determined by the TUNEL assay using an In Situ Cell Death Detection Kit, TMR red (Roche).

Cell Culture. Mouse EC isolation was performed as described in the Supplemental Data. HAEC (Clonetics) and HUVEC (ATCC) cells were grown in EC growth medium (EGM) (Clonetics/Cambrex). For FGF-2 or VEGF treatment, ECs were starved with EC basal medium (EBM-2) with 0.1% FBS for 24 hr, and then treated with growth factors for the indicated periods of time. Adenovirus expressing miR-126 or lacZ was generated and cells were infected as described (Wang et al., 2008). Retrovirus expressing Spred-1 or GFP was generated as described (Nonami et al., 2004). For miR-126-3p inhibitor transfection, HAEC cells were transfected with 2′-O-methyl-miR-126 antisense oligonucleotide (Ambion) and/or human Spred-1 siRNA pool, or a control oligonucleotide at a concentration of 50 nM using Liptofectamine 2000 (Invitrogen). Forty-eight hr after transfection, the cells were starved, treated with VEGF-A, and harvested for protein analyses. miR-126 expression was determined by northern blot analysis with miR-126-3p Starfire™ probe. For aortic ring virus infection, Spred-1 or control GFP retrovirus was added to the cultured aortic rings. Transfection of the mouse Spred-1 siRNA pool into miR-126 null aortic rings was performed as described above. After overnight transfection or infection, the aortic rings were cultured in fresh medium for 4-6 days to monitor the aortic ring sprouting. The expression of Spred-1 was determined by western blot and real-time PCR analysis.

Reporter Assays. The 0.5 kb region 1 enhancer of pEgfl7/miR-126, and the ETS DNA binding site deletion mutant of the fragment generated by site-directed mutagenesis, was cloned into the pGL3 vector upstream of an engineered ANF basal promoter. COS-7 cells in 24-well plates were transfected with 50 ng of reporter plasmids in the presence or absence of increasing amount of Ets1 or Ets1 DNA-binding mutant expression plasmid (John et al., 2008).

Spred-1 3′ UTR and Spred-1 mutant 3′ UTR generated by mutagenesis were directionally cloned into the pMIR-REPORT vector (Ambion). miR-126 genomic DNA fragment and miR-126 m DNA fragment were cloned into the pCMV-Myc vector. Spred-1 or Spred1m UTR construct was then cotransfected with miR-126 or miR-126 m expression plasmid into COS-7 cells. Reporter assays were performed as described (Chang et al., 2005).

Aortic Ring Assay. Four-well culture dishes (Nunclon Surface, Nunc) were covered with 250 ml of matrigel (Chemicon) and allowed to gel for 15 min at 37° C., 5% CO2. Thoracic aortas were excised from 4-6 week-old mice. Fibro-adipose tissue was dissected away from the aortas, which were then cut into 1 mm rings, rinsed with EGM-2 (Cambrex), placed on matrigel coated wells and covered with additional Matrigel. The aortic rings were cultured in EGM-2 (Cambrex) plus 3% mouse serum (Taconic).

In vivo Matrigel Plug Assay. Growth factor reduced-Matrigel (BD Bioscience) was mixed with heparin (60 U/ml), and FGF-2 (250 ng/ml, R&D) or PBS as control. Matrigel (0.5 ml) was injected subcutaneously into the ventral area of anesthetized mice. The animals were euthanized after 7 days, and the Matrigel plugs were carefully dissected away from the host tissue, and processed for frozen section and immunostained for PECAM1.

Scratch-Wound Assay. The scratch-wound assay was performed using HUVEC cells as described (Wang et al., 2008).

Statistics. Statistics was carried out using 2 way t test. P values less than 0.05 were considered to be significant.

### Example 2

### Results

Endothelial-Specific Expression of miR-126. In light of recent studies implicating miRNAs in cardiovascular development and disease, the inventors searched publicly available databases for miRNAs that appeared to be restricted to cardiovascular tissues. Among several such miRNAs, miR-126 appeared enriched in tissues with a high vascular component, such as heart and lung (Lagos-Quintana et al., 2002). A survey of miRNA expression patterns in zebrafish also showed miR-126 to be specific for the vascular system (Wienholds et al., 2005).

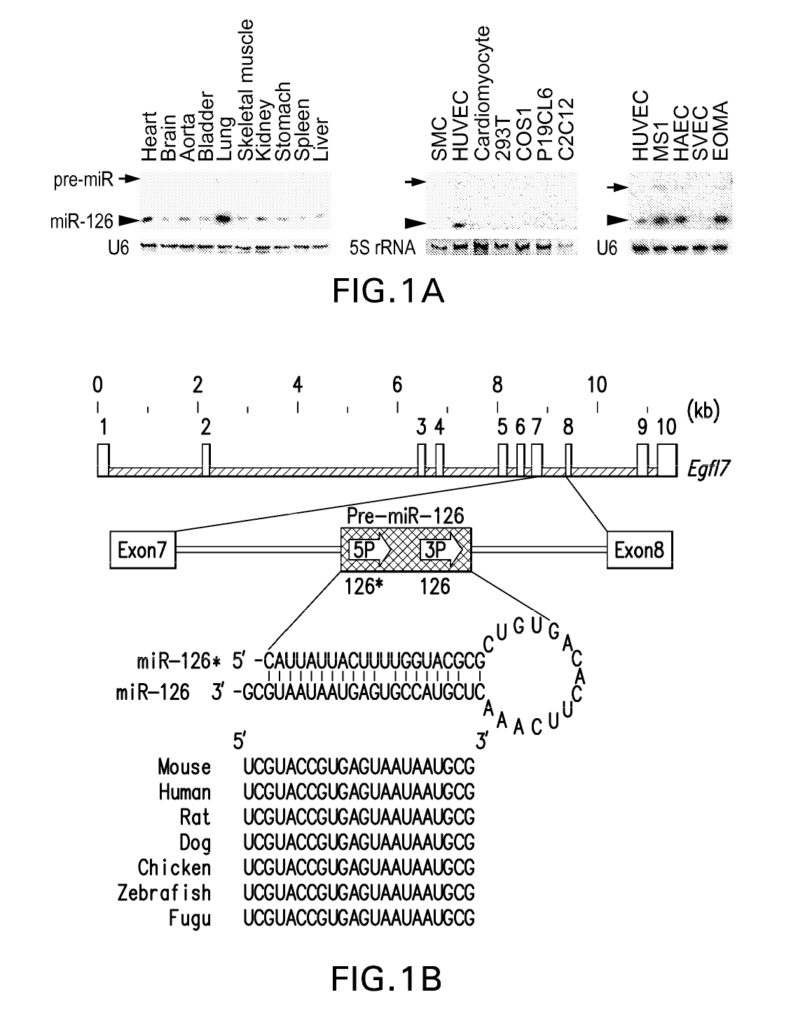

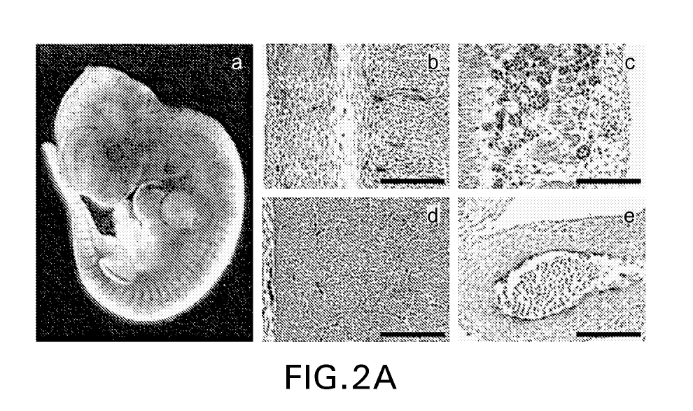

Northern blot analysis showed miR-126 to be expressed in a broad range of tissues, with highest expression in lung and heart (FIG. 1A) consistent with prior studies (Harris et al., 2008; Lagos-Quintana et al., 2002; Musiyenko et al., 2008). miR-126* was detectable at only trace levels of expression (data not shown). A survey of cell lines revealed miR-126 to be expressed in primary human umbilical vein ECs (HUVECs) and in numerous EC cell lines, including the MS1, HAEC, and EOMA cell lines, but not in SV40 transformed ECs (SVECs) or nonendothelial cell types (FIG. 1A).

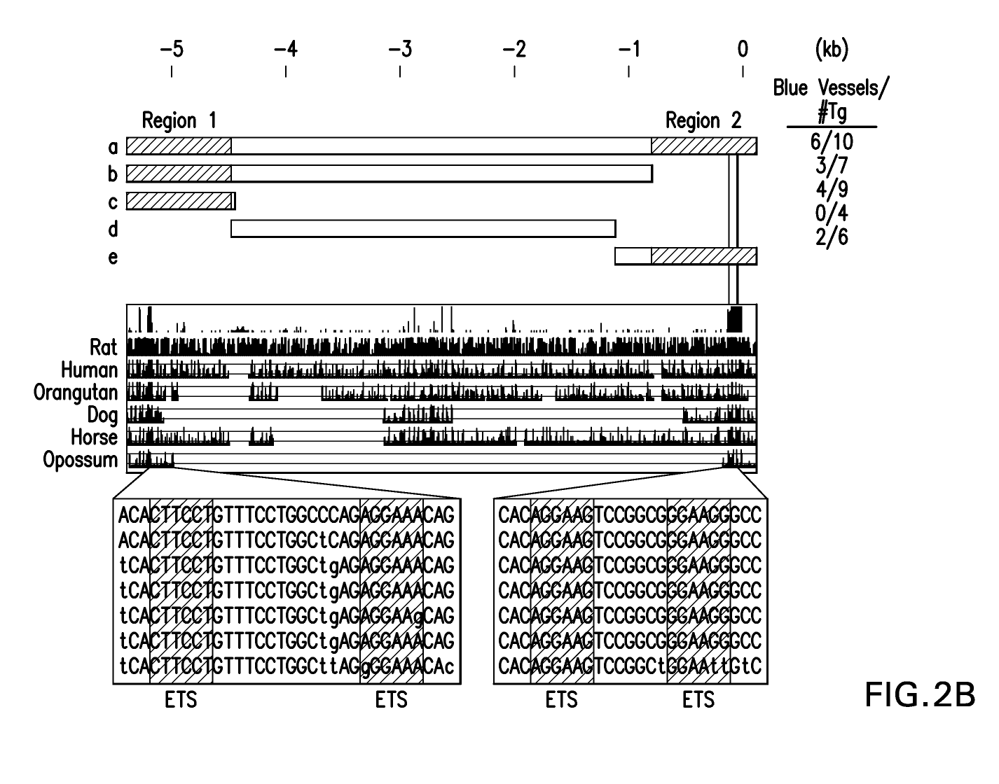

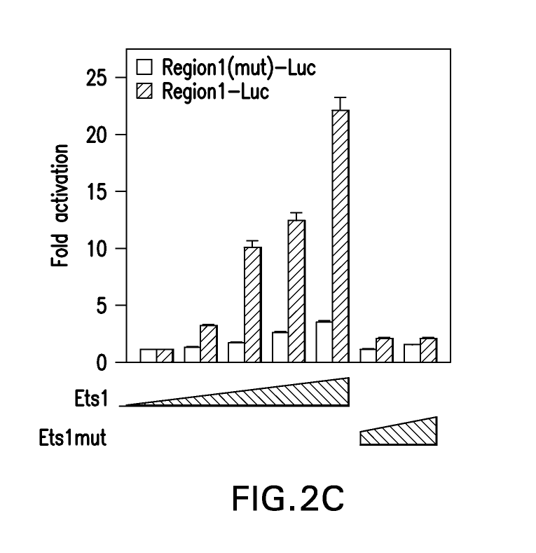

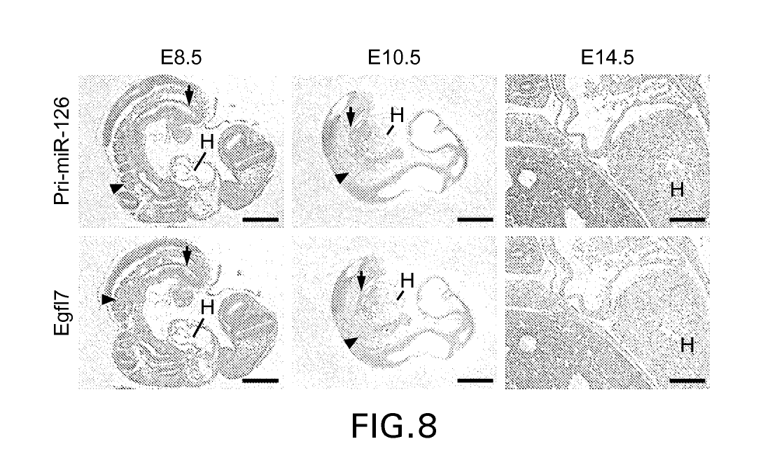

miR-126 (also referred to as miR-126-3p) and miR-126* (miR-126-5p) are conserved from Fugu to Homo sapiens (microrna.sanger.ac.uk/sequences/index.shtml). In mammals and birds, miR-126 and -126* are encoded by intron 7 of the EGF-like domain 7 (Egfl7) gene (FIG. 1B), which encodes an EC-specific secreted peptide that has been reported to act as a chemoattractant and inhibitor of smooth muscle cell migration (Campagnolo et al., 2005; Fitch et al., 2004; Parker et al., 2004; Soncin et al., 2003). The expression pattern of miR-126 in tissues and cell lines parallels that of Egfl7 (Fitch et al., 2004; Soncin et al., 2003), consistent with the conclusion that the miRNA is processed from intronic RNA sequence of the pre-Egfl7 mRNA. RT-PCR using RACE-ready cDNAs from human placenta with primers upstream and downstream of intron 7 of Egfl7 showed that miR-126 was generated from a subset of Egfl7 transcripts in which intron 7 was retained (unpublished data). In situ hybridization with mouse embryo sections using a portion of intron 7 encompassing pri-miR-126 as a probe revealed EC-specific expression of miR-126 from E7.5 to adulthood (FIG. 8), similar to Egfl7. EC-Specific Transcription of Egfl71miR-126 To further visualize the expression pattern of miR-126 in vivo, the inventors cloned 5.4 kb of genomic DNA immediately 50 of the Egfl7/miR-126 gene into a lacZ reporter gene and generated transgenic mice. This DNA fragment was sufficient to direct expression specifically in ECs throughout embryogenesis and in adult tissues (FIG. 2A and data not shown).

The 5.4 kb DNA fragment contained two regions (Region 1 and Region 2) of high evolutionary conservation, each of which was sufficient to direct endothelial-specific expression in vivo (FIG. 2B). Both regions contained conserved consensus sequences for binding of Ets transcription factors, which have been implicated in endothelial-specific transcription (Lelievre et al., 2001). Ets1 potently transactivated these regulatory regions in transfected COS-7 cells, whereas an Ets1 mutant lacking the DNA binding domain was devoid of activity. Moreover, mutations in the Ets sites blunted transcriptional activation by Ets1 (FIG. 2C) and abolished expression of the lacZ transgene in ECs in vivo (data not shown). These findings suggest that Ets transcription factors are sufficient and necessary for endothelial-specific transcription of Egfl71miR-126.

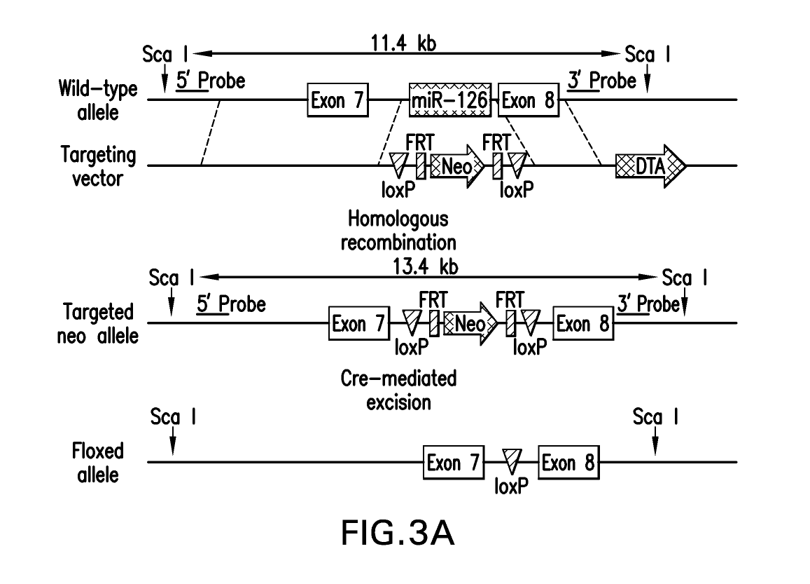

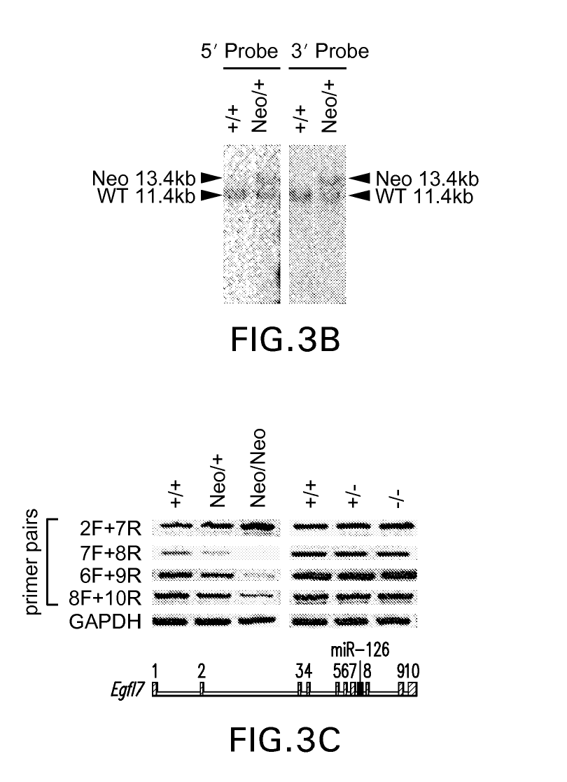

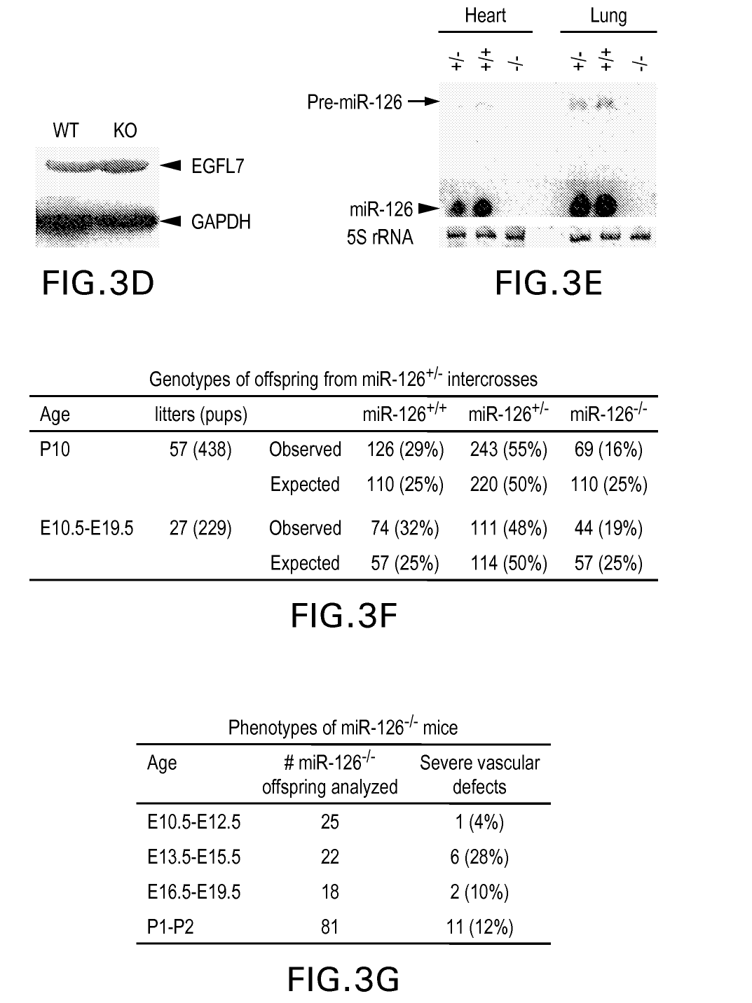

Creation of miR-126 Null Mice. To explore the functions of miR-126 in vivo, the inventors deleted the region of intron 7 of the Egfl7 gene encoding miR-126 and inserted a neomycin-resistance cassette flanked by loxP sites (FIGS. 3A and 3B). Mice heterozygous for the mutant miR-126 allele were intercrossed to obtain miR-126neo/neo mutants. The presence of the neomycin cassette in the Egfl7 intron altered the splicing of surrounding exons, as detected by RT-PCR (FIG. 3C, left panel). Removal of the neomycin cassette from the Egfl7 intron by breeding miR-126^{neo/+} mice to mice expressing Cre recombinase under control of the CAG promoter normalized the transcription and splicing of Egfl7 (FIG. 3C, right panel).

miR-126^{+/-} mice were intercrossed to obtain miR-126^{-/-} mice. Neither the mature miR-126 nor the stem loop was expressed in these animals (FIG. 3E). The targeted mutation did not alter the expression of Egfl7 mRNA (FIG. 3C, right panel) or EGFL7 protein (FIG. 3D) in tissues from homozygous mutant mice.

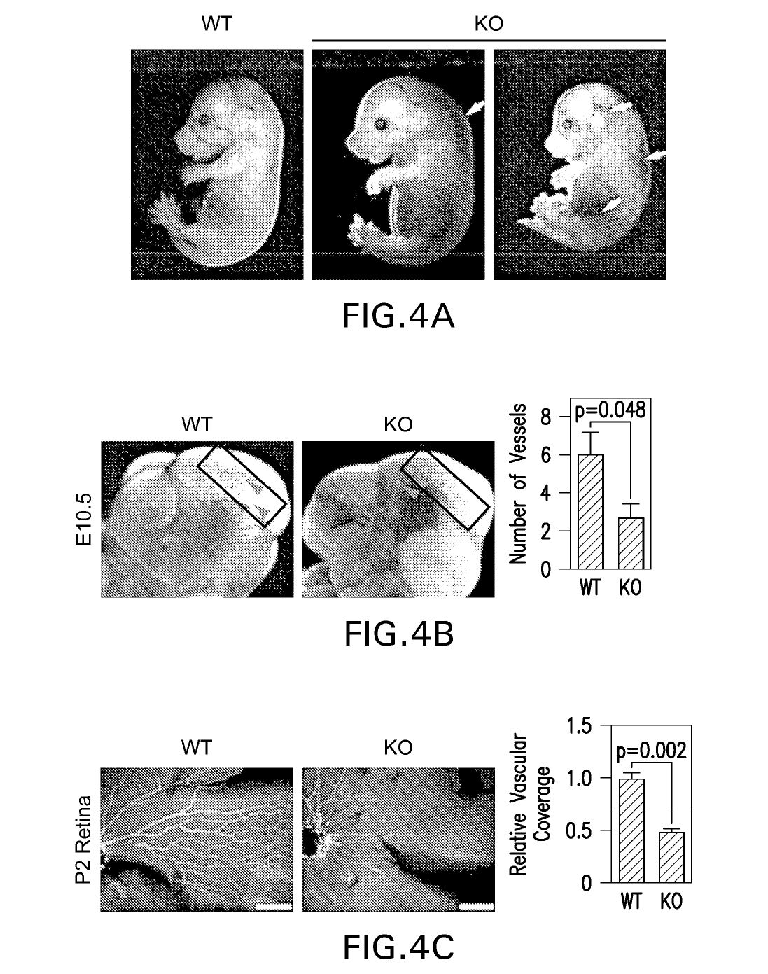

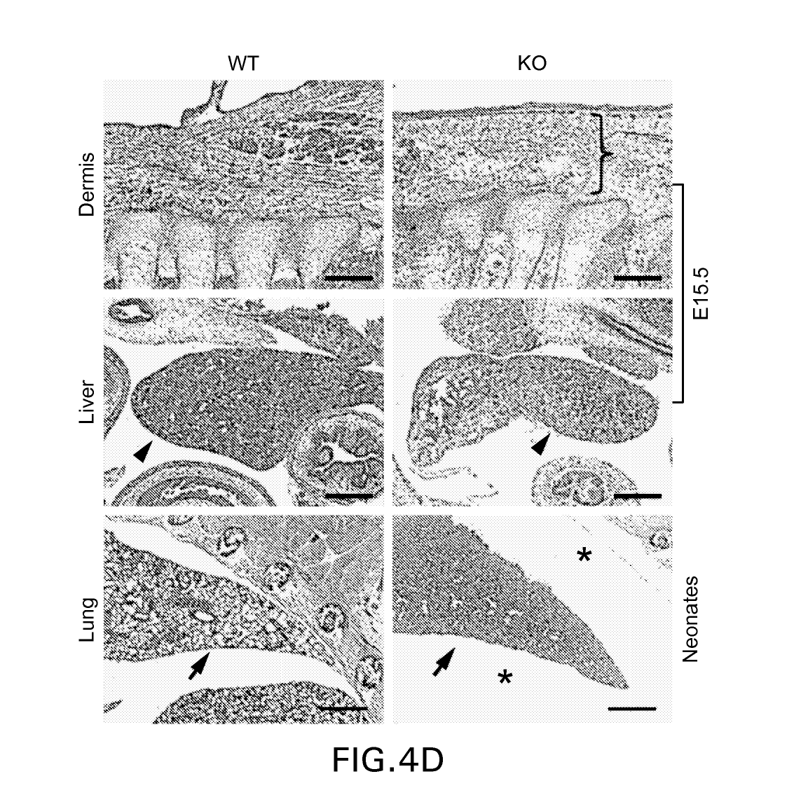

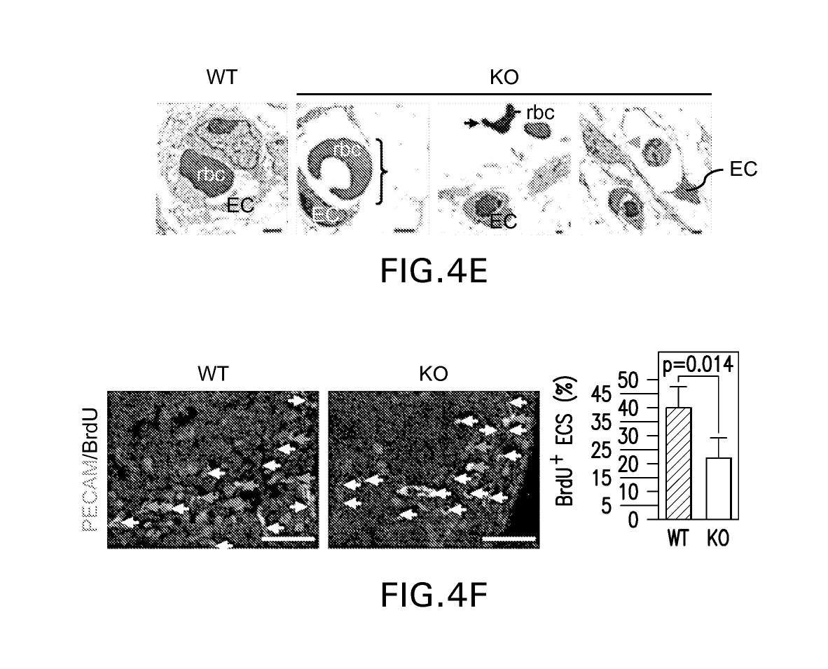

Vascular Abnormalities in miR-126 Mutant Mice. miR-126^{-/-} mice were obtained at a lower than predicted frequency from miR-126 intercrosses (FIG. 3F). At post-natal day 10 (P10), 16% of offspring obtained from heterozygous intercrosses were homozygous mutants, versus the expected 25%. Thus, about 40% of the miR-126^{-/-} mice died embryonically or perinatally. Analysis of embryos obtained from timed matings revealed miR-126^{-/-} embryos that were dead or dying with severe systemic edema, multifocal hemorrhages, and ruptured blood vessels throughout embryogenesis (FIGS. 4A and 4B). The highest percentage of embryos with vascular abnormalities was observed from E13.5 to E15.5 (FIG. 3G). However, a failure in growth of the cranial vessels was observed as early as E10.5 (FIG. 4B), prior to systemic edema, hemorrhage, or overall embryo demise, indicating that the vascular defects represent a primary effect of miR-126 deletion. Similarly, vascularization of the retina, which begins at P0 and involves the outward migration of ECs from the central retinal artery, was severely impaired in miR-126^{-/-} mice (FIG. 4C) in the absence of other morphological abnormalities.

Histological analysis of mutant embryos and neonates showed abnormal thickening of the epidermis, a hallmark of edema, with erythrocytes and inflammatory cells in the tissue spaces, as well as congestion of red blood cells in the liver, which may reflect compensatory erythropoiesis caused by hypoxia (FIG. 4D). Of the miR-126^{-/-} mice that survived to birth, approximately 12% died by P1 and contained excessive protein-rich fluid in the pleural spaces of the thoracic cavity, an indication of severe edema. The lungs were also not inflated, possibly secondary to the severe edema (FIG. 4D). Edema and hemorrhage were also observed in the thoracic cavity outside of the pericardial space in some miR-126^{-/-} newborn mice. These abnormalities suggested a role for miR-126 in maintenance of endothelial integrity. Indeed, electron microscopy of miR-126^{-/-} embryos confirmed the lack of endothelial integrity and revealed extensive rupture of blood vessels and lack of tight cell-cell interactions (FIG. 4E).

Platelet/endothelial cell adhesion molecule (PECAM)-positive ECs from vascularized tissues of miR-126^{-/-} embryos at E15.5 displayed diminished proliferation compared to wild-type ECs, as detected by BrdU staining (FIG. 4F), whereas proliferation of non-ECs was not significantly different in wild-type and mutant embryos (data not shown). The inventors detected no difference in apoptosis between wild-type and mutant ECs at E15.5 by TUNEL staining (data not shown).

The surviving miR-126^{-/-} mice appeared normal to adulthood and displayed no obvious abnormalities based on histological analysis of tissues. Male mutant mice were fertile. However, females were subfertile with reduced litter size (data not shown). The inventors conclude that miR-126 plays an important role in maintenance of vascular integrity during embryogenesis, but is not essential for vascular homeostasis after birth.

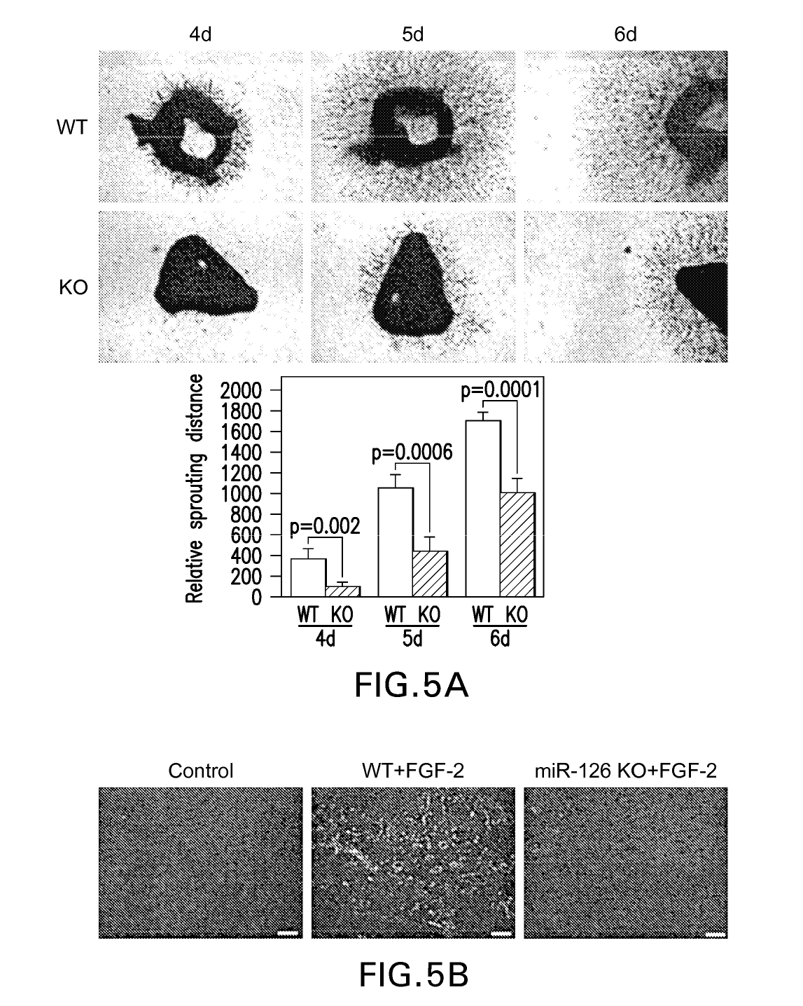

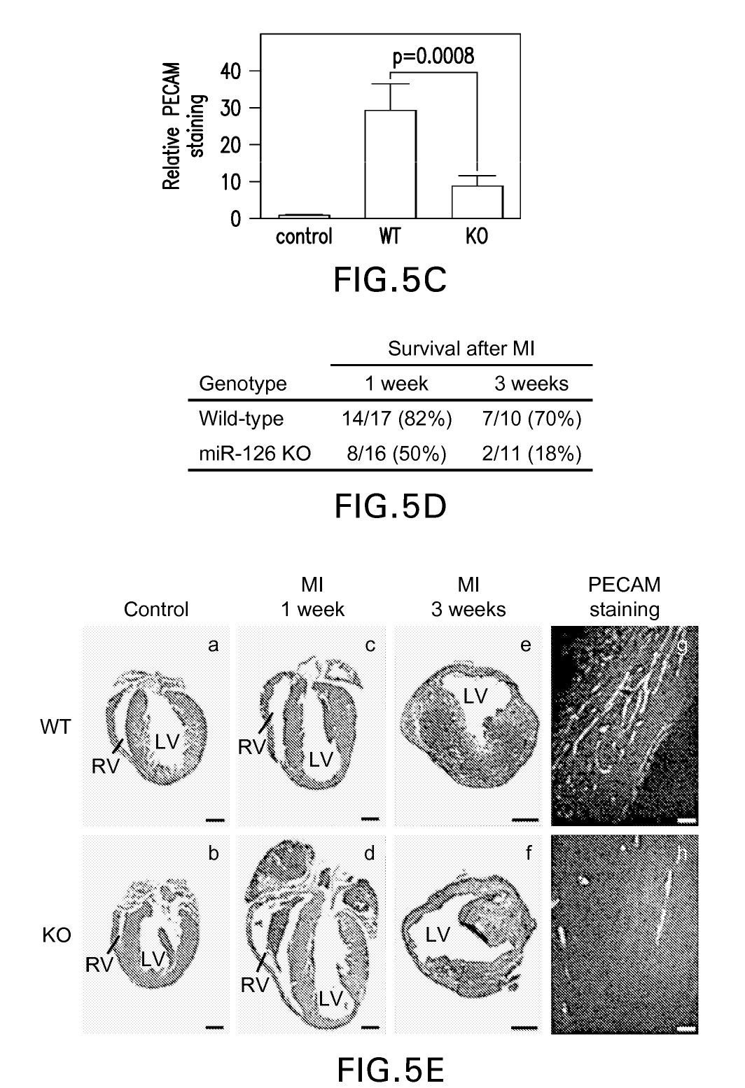

Defective Angiogenesis of miR-126^{-/-} ECs. To explore the angiogenic functions of miR-126^{-/-}, the inventors analyzed sprouting angiogenesis using an ex vivo aortic ring assay. Aortic rings from 4-week old miR-126^{-/-} mice and wild-type littermates were isolated and cultured on matrigel with endothelial growth medium containing FGF-2 and VEGF and supplemented with 3% mouse serum. ECs from wild-type mice showed extensive outgrowth between days 4 and 6 of culture, whereas endothelial outgrowth was dramatically impaired in aortic rings obtained from miR-126^{-/-} mice (FIG. 5A). Staining for PECAM confirmed the identity of ECs in aortic ring cultures (data not shown).

The inventors further analyzed the angiogenic response of ECs in miR-126^{-/-} mice in vivo using a matrigel plug EC invasion assay in which mice were injected subcutaneously with a matrigel plug containing the proangiogenic factor FGF-2, or PBS as control. In response to angiogenic growth factor signaling, ECs typically migrate into the matrigel plug and assemble into a primitive vascular network, which can be detected by PECAM staining 1 week later. EC invasion requires angiogenic growth factors and is not observed in PBS control matrigel plugs. ECs from miR-126^{-/-} mice showed a dramatically diminished angiogenic response to FGF-2 compared to controls (FIGS. 5B and 5C).

Reduced Survival of miR-126^{-/-} Mice Following Myocardial Infarction. The diminished angiogenic response of miR-126^{-/-} ECs revealed in the matrigel EC invasion assay suggested that miR-126 might play an important role in neoangiogenesis of adult tissues, as occurs in response to injury. Neoangiogenesis is essential for cardiac repair following myocardial infarction (MI), when collateral vessels form at the site of the infarct to maintain blood flow to ischemic cardiac tissue (Kutryk and Stewart, 2003). Myocardial vascularization following MI requires signaling by VEGF and FGF (Scheinowitz et al., 1997; Syed et al., 2004).

The inventors therefore compared the response of wild-type and miR-126 null mice to MI following surgical ligation of the left coronary artery. MI in wild-type mice typically results in an infarct, followed by the formation of a scar. Under the surgical conditions for these experiments, 70% of wild-type mice survived for at least 3 weeks following MI (FIG. 5D). In contrast, half of miR-126^{-/-} mice died by 1 week post-MI, and nearly all died by 3 weeks (FIG. 5D).

Unoperated hearts from wild-type and miR-126 mutant mice were indistinguishable histologically (FIG. 5E, panels a and b). One week after MI, mutant mice showed ventricular dilatation compared to wild-type hearts and commonly developed atria thrombi (FIG. 5E, panels c and d), indicative of heart failure. By 3 weeks post-MI, histological analysis showed more extensive fibrosis and loss of functional myocardium in miR-126 mutants compared to wild-type controls (FIG. 5E, panels e and f). Many miR-126 mutant animals that died during this period also displayed ventricular rupture, a known consequence of inadequate myocardium that can result from deficient blood flow (data not shown), whereas myocardial rupture was never observed in wild-type mice following MI.

PECAM staining revealed extensive vascularization of the injured myocardium in wild-type mice 3 weeks following MI. In contrast, there was a relative paucity of new vessels in the mutants, and those vessels that were observed appeared truncated and fragmentary (FIG. 5E). Thus, miR-126 appears to be important for normal neovascularization following MI.

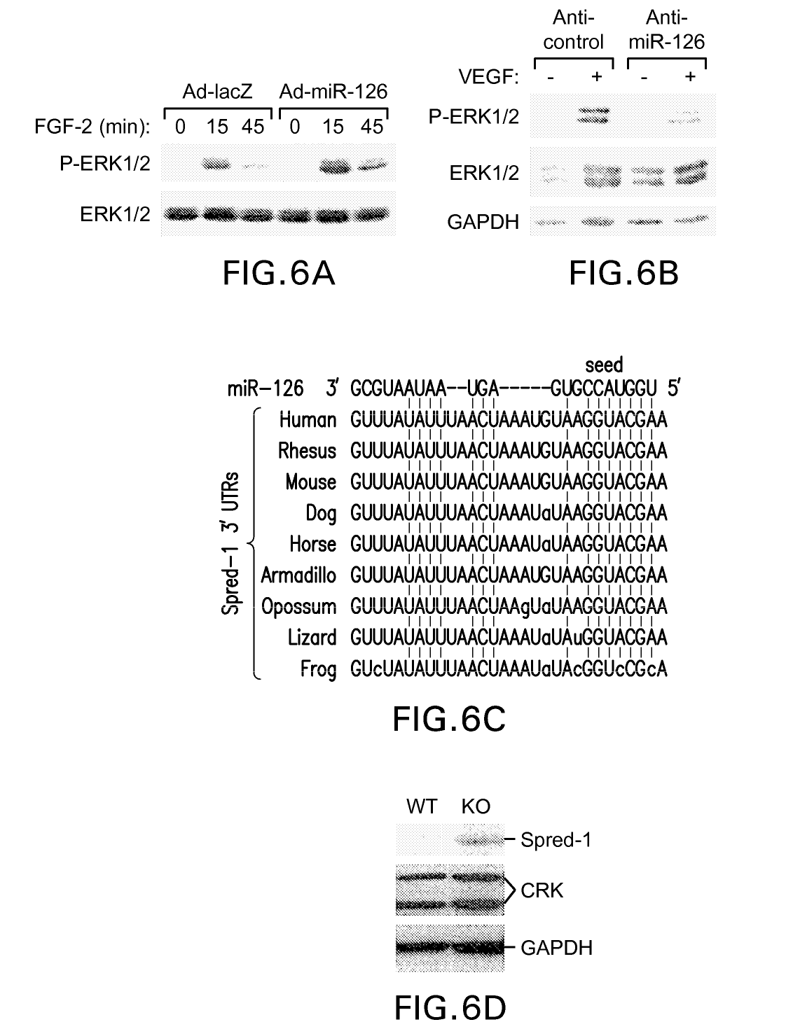

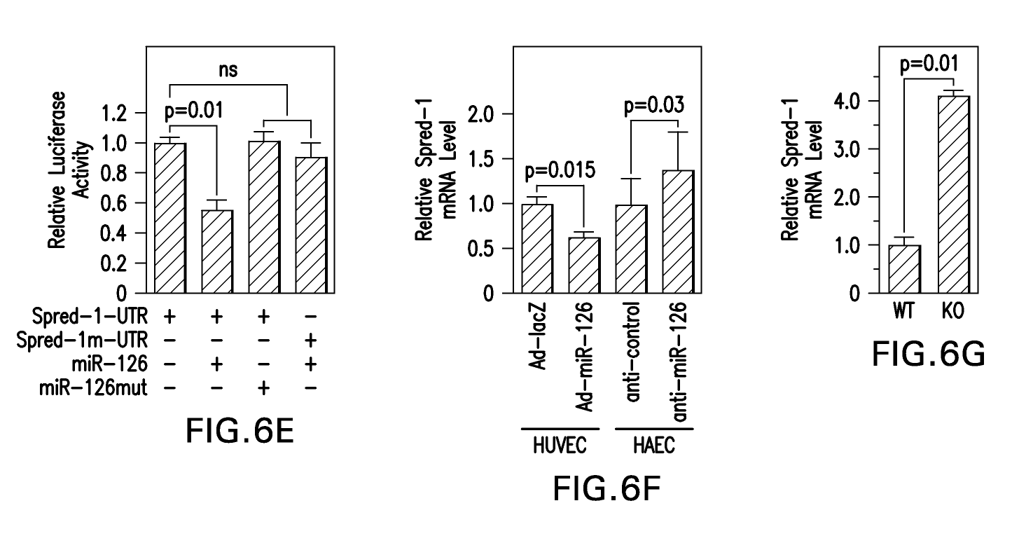

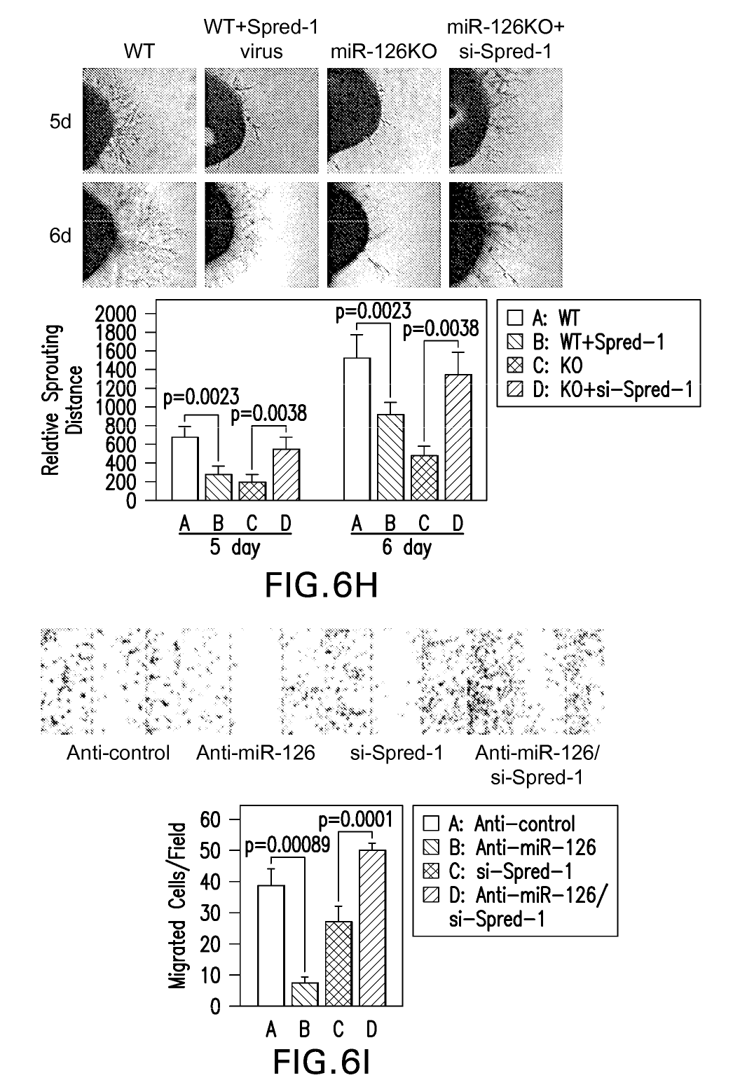

Modulation of Angiogenic Signaling by miR-126. Vascular defects in miR-126^{-/-} embryos, combined with the impaired angiogenic activity of mutant ECs, suggested that miR-126 was essential for normal responsiveness of ECs to angiogenic growth factors. To further test this possibility, the inventors infected HUVECs with a miR-126 expressing adenovirus (Ad-miR-126) and examined MAP kinase activation by FGF-2, as detected by phosphorylation of ERK1/2. As shown in FIG. 6A, activation of ERK1/2 phosphorylation by FGF-2 was enhanced approximately 2-fold by Ad-miR-126 compared to an Ad-lacZ control. Conversely, knockdown of miR-126 expression with a 20-O-methyl-miR-126 antisense oligonucleotide diminished ERK phosphorylation in response to VEGF, compared to a control oligonucleotide (FIG. 6B). These findings suggested that miR-126 augments MAP kinase pathway activation by FGF and VEGF.

Inhibition of Spred-1 Expression by miR-126. To identify potential mRNA targets of miR-126 that might contribute to the endothelial abnormalities of miR-126 mutant mice, the inventors compared the gene expression profiles by microarray analysis of ECs isolated from adult kidneys of wild-type and miR-126 null mice. Since most miRNAs promote the degradation of their target mRNAs (Jackson and Standart, 2007), the inventors focused on mRNAs that were upregulated in miR-126^{-/-} ECs (Table 3). Numerous mRNAs involved in angiogenesis, cell adhesion, inflammatory/cytokine signaling, and cell cycle control were upregulated in miR-126^{-/-} EC cells. Among this group of transcripts, the inventors identified three mRNAs that were also predicted by various miRNA target prediction programs to be evolutionarily conserved targets of miR-126 (Table 4): Sprouty-related protein-1 (Spred-1), VCAM-1, and integrin α-6. Indeed, VCAM-1 mRNA was recently shown to be a target for repression by miR-126 in vitro (Harris et al., 2008).

TABLE 3
Dysregulated Genes in miR-126^{-/-} Endothelial Cells

| | | | Fold | Predicted Target |
|---|---|---|---|---|
| Category | Dys-regulated Genes in isolated endothelial cells miR-126 KO | | increase | (Y or N) |
| Angiogenesis | Sos1 | Son of sevenless | 28.7 | |
| | Cryab | crystallin, alpha B | 16.3 | |
| | Pdgfra | platelet derived growth factor receptor, alpha polypeptide | 11.8 | |
| | Pik3c2a | Phosphatidylinositol 3 kinase | 3.8 | |
| | Stat1 | Signal Transducer and Activator of Transcription 1 | 3.7 | |
| | Frzb | frizzled-related protein | 3.5 | |
| | Efnb2 | ephrin B2 | 2.7 | |
| | Spred1 | sprouty protein with EVH-1 domain 1, related sequence | 2 | Y |
| | Tek | endothelial-specific receptor tyrosine kinase | 1.8 | |
| | Braf | Braf transforming gene | 1.7 | |
| | Yes1 | Yamaguchi sarcoma viral oncogene homolog | 1.6 | |
| Cell adhesion | Pcdh20 | protocadherin 20 | 14.6 | |
| | Pcdh21 | protocadherin 21 | 13.6 | |
| | Cdh10 | cadherin 10 | 6.1 | |
| | Vcam-1 | Vascular cell adhesion molecule-1 | 3.6 | Y |
| | Celsr1 | cadherin EGF LAG seven-pass G-type receptor 1 | 3.2 | |
| | Fat4 | FAT tumor suppressor homolog 4 (Drosophila) | 3 | |
| | 2610005L07Rik | Cadherin 11 | 1.8 | |
| | Yes1 | Yamaguchi sarcoma viral oncogene homolog | 1.6 | |
| Inflammatory/ | Sos1 | Son of sevenless | 28.7 | |
| Cytokine pathway | Vav3 | Vav oncogene | 20.4 | |
| | Dcamk1 | doublecortin and Ca2+ calmodulin dependent protein | 18.7 | |
| | | kinase-like 1 | | |
| | Plcb4 | Phospholipase C, beta 4 | 6.8 | |
| | Gng4 | Guanine nucleotide binding protein | 5.2 | |
| | NFAT5 | Nuclear factor of activated T-cells 5 | 4.2 | |
| | Ccl7 | Chemokine ligand 7 | 3.9 | |
| | Pik3c2a | Phosphatidylinositol 3 kinase | 3.8 | |
| | Itpr2 | Inositol 1,4,5-triphosphate receptor 2 | 3.7 | |
| | IL6 | Interleukin 6 | 3.7 | |
| | Stat1 | Signal Transducer and Activator of Transcription 1 | 3.7 | |
| | Gng5 | Guanine nucleotide binding protein | 2.8 | |
| | Ccl2 | Small inducible cytokine A | 2.8 | |
| | Itga6 | Integrin alpha-6 | 2.6 | Y |
| | Camk2d | Calcium/calmodulin-dependent protein kinase II, delta | 2.4 | |
| | Gnaq | Guanine nucleotide binding protein, alpha q polypeptide | 2.2 | |
| | Braf | Braf transforming gene | 1.7 | |
| | Rock1 | Rho-associated coiled-coil containing protein kinase 1 | 1.7 | |
| Cell cycle control | Kit | Kit oncogene | 21.5 | |
| and oncogenesis | Vav3 | Vav 3 oncogene | 20.4 | |
| | Mybl1 | Myeloblastosis oncogene like 1 | 13.3 | |
| | PDGFra | Platelet derived frowth factor receptor, alpha | 11.8 | |
| | Ctsb | Cathepsin B | 8.5 | |
| | pms1 | Postmeiotic segregation increased 1 | 8 | |
| | TAF15 | TAF15 RNA polymerase 11 | 4.7 | |
| | Irf7 | Interferon regulatory factor 7 | 3.8 | |
| | smad7 | Mad homolog 7 | 3.5 | |
| | Pgr | Progesterone receptor | 3.4 | |
| | Srgap2 | Slit-Robo Rho GTPase activating protein 2 | 2.9 | |
| | Phf17 | PHD finger protein 17 | 2.1 | |
| | Usp6nl | USP6 N-terminal like | 1.9 | |
| | Smad1 | Mad homolog 1 | 1.8 | |
| | Phf14 | PHD finger protein 14 | 1.8 | |
| | Braf | Braf transforming gene | 1.7 | |
| | Rock1 | Rho-associated coiled-coil containing protein kinase 1 | 1.7 | |
| | Brca 1 | Breast cancer 1 | 1.6 | |
| | Yes1 | Yamaguchi sarcoma viral oncogene homolog | 1.6 | |
| | Mrg1 | Myeloid ecotropic viral integration site-related gene 1 | 1.5 | |

Dys-regulated genes in miR-126^{-/-} kidney endothelial cells as determined by microarray analysis (cut-off: 1.5 fold).
Up-regulated genes are grouped in categories that were overrepresented in the asray as determined by PANTHER (www.pantherdb.org/tools/).
— fold increase is shown.
Y indicates that the gene is a predicted target of miR-126.
Of note, some genes are listed in multiple categories.

TABLE 4
Conserved Targets of miR-126

| Gene | | |
|---|---|---|
| Symbol | Description | Validation |
| TOM1 | Target of myb1 (chicken) | |
| SLC7A5 | Solute carrier family (Cationic amino acid | |
| | transporter, y+ system), member 5 | |
| RGS3 | Regulator of G-protein signallin 3 | |
| PTPN9 | Protein tyrosine phosphatase, non-receptor type 9 | |
| PLK2 | Polo-like kinase 2 (Drosophila) | |
| GOLPH3 | Golgi phosphoprotein 3 (coat protein) | |
| FBXO33 | F-box protein 33 | |
| CRK | V-crk sarcoma virus CT10 oncogene homolog | |
| | (avian) | |
| SPRED1 | Sprouty-related, EVH1 domain containing 1 | Yes |
| PHF15 | PhD finger protein 15 | |
| ITGA6 | Integrin alpha 6 | |
| IRS1 | Insulin receptor substrate 1 | |
| VCAM1 | Vascular cell adhesion protein 1 precursor | Yes |
| | (V-CAM 1) | |
| CD97 | CD97 antigen | Yes |

The miR-126 target genes were bioinformatically predicted by TargetScan Version 4.1 (www.targetscan.org/), PicTar (pictar.bio.nyu.edu/) and/or Mirand (microrna.sanger.ac.uk/targets/v5/).
In the validation column, “Yes” indicates that the targets were experimentally validated in miR-126^{-/-} endothelial cells.

Intriguingly, Spred-1 has been shown to function as a negative regulator of the Ras/MAP kinase pathway (Wakioka et al., 2001). Given the ability of miR-126 to enhance MAP kinase signaling in response to VEGF and FGF, and the diminished angiogenic growth factor signaling in the absence of miR-126, Spred-1 seemed a likely mediator of the angiogenic actions of miR-126. The predicted energy of the miR-126/Spred-1 interaction is ˜−17.9 kcal/mol. Most importantly, the “seed” region (nucleotides 1-7) of miR-126 is completely complementary to the sequence of the Spred-1 3′ UTR, and the complementary sequences of miR-126 and the Spred-1 30 UTR are conserved from amphibians to mammals (FIG. 6C). Consistent with the conclusion that Spred-1 mRNA is a target for repression by miR-126, Spred-1 protein expression was increased in yolk sac from miR-126^{-/-} mice compared to wild-type littermates (FIG. 6D). In contrast, CT10 regulator of kinase (CRK), which is predicted by several miRNA target prediction programs to be a miR-126 target, was unchanged, as was GAPDH, as a control.

When the Spred-1 3′ UTR was fused to a luciferase reporter and tested for repression by miR-126 in transfected cells, miR-126 strongly repressed expression of the Spred-1 3′ UTR luciferase reporter (FIG. 6E). Mutation of six nucleotides in the miR-126 “seed” region (miR-126 m) or its complementary sequence in the Spred-1 3′ UTR (Spred-1 m UTR) relieved the repressive effect of miR-126 (FIG. 6E). Infection of HUVEC cells with an adenovirus expressing miR-126 also repressed expression of Spred-1 mRNA by about 2-fold (FIG. 6F). Conversely, a miR-126 antisense RNA elevated the expression of Spred-1 mRNA in HAEC cells (FIG. 6F). The efficiency of miR-126 overexpression or knockdown was monitored by northern blot analysis with miR-126 probe (data not shown).

To establish whether miR-126 is necessary to repress Spred-1 expression, ECs were isolated from the kidneys of miR-126^{-/-} and wild-type adult mice. The identity of ECs was monitored by the uptake of DiI-labeled acetylated low density lipoprotein (DiI-Ac-LDL) and staining with an antibody against von Willebrand factor (data not shown). As expected, Spred-1 mRNA was significantly upregulated in miR-126^{-/-} ECs compared to wild-type ECs (FIG. 6G), confirming the microarray results.

Further support for the involvement of Spred-1 in inhibiting miR-126-modulated EC migration and angiogenesis, was provided by the aortic ring assay in which retrovirus-mediated overexpression of Spred-1 diminished EC outgrowth (FIG. 6H), whereas knockdown of Spred-1 expression with a small interfering RNA enhanced endothelial outgrowth in explants from miR-126^{-/-} mice (FIG. 6H). Finally, in a scratch-wound assay in vitro, miR-126 antisense RNA dramatically impaired HUVEC migration, whereas Spred-1 siRNA restored migratory activity to cells expressing miR-126 antisense RNA (FIG. 6I). These results support the conclusion that miR-126 augments angiogenic signaling by diminishing the inhibitory influence of Spred-1 on the MAP kinase pathway.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.
U.S. Pat. No. 4,873,191
U.S. Pat. No. 5,844,107
U.S. Pat. No. 5,877,302
U.S. Pat. No. 5,972,900
U.S. Pat. No. 5,972,901
U.S. Pat. No. 6,008,336
U.S. Pat. No. 6,077,835
U.S. Pat. No. 6,200,801
U.S. Pat. No. 6,693,187
U.S. Pat. No. 6,838,283
U.S. Pat. No. 7,067,641
U.S. Publn. 20020150626
U.S. Publn. 20030032615
U.S. Publn. 20030203865
U.S. Publn. 20040048787
Ambros, Cell, 113(6):673-676, 2003.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Baichwal and Sugden, In: Gene Transfer, Kucherlapati (Ed.), Plenum Press, NY, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-11517, 1997.
Bartel, Cell, 116:281-297, 2004.
Benvenisty and Neshif, Proc. Natl. Acad. Sci. USA, 83(24):9551-9555, 1986.
Berkhout et al., Cell, 59:273-282, 1989.
Bhaysar et al., Genomics, 35(1):11-23, 1996.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Brennecke et al., Cell, 113:25-36, 2003.
Brinster et al., Proc. Natl. Acad. Sci. USA, 82(13):4438-4442, 1985.
Bulla and Siddiqui, J. Virol., 62:1437, 1986.
Calin et al., Proc. Natl. Acd. Sci. USA, 99:15524-15529, 2002.
Campagnolo et al. Am. J. Pathol., 167:275-284, 2005.
Campbell and Villarreal, Mol. Cell. Biol., 8:1993, 1988.
Campere and Tilghman, Genes and Dev., 3:537, 1989.
Campo et al., Nature, 303:77, 1983.
Care et al., Nat. Med. 13:613-618, 2007.
Carmeliet, Nat. Med., 9:653-660, 2003.
Carrington et al. Science, 301(5631):336-338, 2003.
Celander and Haseltine, J. Virology, 61:269, 1987.
Celander et al., J. Virology, 62:1314, 1988.
Chandler et al., Cell, 33:489, 1983.
Chang and Karin, Nature, 410(6824):37-40, 2001.
Chang et al., Biochim. Biophys. Acta, 1092(2):153-160, 1991.
Chang et al., Cell, 126:321-334, 2006.
Chang et al., Mol. Cell. Biol., 9:2153, 1989.
Chang et al., Nature, 430(7001):785-789, 2004.
Chang et al., Proc. Natl. Acad. Sci. USA, 102:8120-8125, 2005.
Chatterjee et al., Proc. Natl. Acad. Sci. USA, 86:9114, 1989.
Chen and Okayama, Mol. Cell Biol., 7(8):2745-2752, 1987.
Chen et al., Science, 303(5654):83-86, 2004.
Choi et al., Cell, 53:519, 1988.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Cross and Claesson-Welsh, Trends Pharmacol. Sci., 22:201-207, 2001.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
Eichhorn and Bristow, Circulation, 94:2285-2296, 1996.
Eliceiri et al., J. Cell Biol., 140:1255-1263, 1998.
EPO 0273085
Fatkin et al., J. Clin. Invest., 106(11):1351-1359, 2000.
Fechheimer, et al., Proc Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fitch et al., Dev. Dyn., 230:316-324, 2004.
Foecking and Hofstetter, Gene, 45(1):101-105, 1986.
Fraley et al., Proc. Natl. Acad. Sci. USA, 76:3348-3352, 1979.
Franz et al., Cardioscience, 5(4):235-43, 1994.
Friedman et al., Genes Devel., 3:1314, 1989.
Fujita et al., Cell, 49:357, 1987.
Garzon et al., Proc. Natl. Acad. Sci. USA, 103:5078-5083, 2006.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Giroux et al., Curr. Biol., 9:369-372, 1999.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol. 15(12):7081-7090, 1995.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. Gen. Virl., 36(1):59-74, 1977.
Greene et al., Immunology Today, 10:272, 1989
Grishok et al., Cell, 106:23-34, 2001.
Grosschedl and Baltimore, Cell, 41:885, 1985.
Grunhaus and Horwitz, Seminar in Virology, 3:237-252, 1992.
Harland and Weintraub, J. Cell Biol., 101(3):1094-1099, 1985.
Harris et al., Proc. Natl. Acad. Sci. USA, 105:1516-1521, 2008.
Haslinger and Karin, Proc. Natl. Acad. Sci. USA, 82:8572, 1985.
Hauber and Cullen, J. Virology, 62:673, 1988.
Hayashi et al., J. Clin. Invest., 113:1138-1148, 2004.
He and Hannon, Nat. Rev. Genet., 5:522-531, 2004.
Heineke and Molkentin, Nat. Rev. Mol. Cell Biol. 7:589-600, 2006.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Hirochika et al., J. Virol., 61:2599, 1987.
Hirsch et al., Mol. Cell. Biol., 10:1959, 1990.
Holbrook et al., Virology, 157:211, 1987.
Hood et al., Science, 296:2404-2407, 2002.
Horlick and Benfield, Mol. Cell. Biol., 9:2396, 1989.
Horwich et al. J. Virol., 64:642-650, 1990.
Huang et al., Cell, 27:245, 1981.
Hug et al., Mol. Cell. Biol., 8:3065, 1988.
Hutvagner et al., PLoS Biol., 2(4):E98, 2004.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jackson and Standart, Sci. STKE, 367:re1-re13, 2007.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
John et al., J. Biol. Chem., 283:951-962, 2008.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Katinka et al., Nature, 290:720, 1981.
Kato et al, J. Biol. Chem., 266:3361-3364, 1991.
Kawamoto et al., Mol. Cell. Biol., 8:267, 1988.
Kelly et al., J. Cell Biol., 129(2):383-396, 1995.
Kiledjian et al., Mol. Cell. Biol., 8:145, 1988.
Kimura et al., Dev. Growth Differ. 39(3):257-265, 1997.
Kiriazis and Kranias, Annu. Rev. Physiol., 62:321-351, 2000.
Klaassen's The Pharmacological Basis of Therapeutics
Klamut et al., Mol. Cell. Biol., 10:193, 1990.
Klein et al., Nature, 327:70-73, 1987.
Koch et al., Mol. Cell. Biol., 9:303, 1989.
Kocher et al., Nat. Med., 7:430-436, 2001.
Kothary et al., Development, 105:707-714, 1989.
Krenz and Robbins, J. Am. Coll. Cardiol., 44:2390-2397, 2004.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983a.
Kriegler et al., Cell, 38:483, 1984.
Kriegler et al., Cell, 53:45, 1988.
Kriegler et al., In: Gene Expression, Alan Liss (Ed.), Hamer and Rosenberg, New York, 1983b.
Krützfeldt et al., Nature, 438:685-689, 2005.
Kuehbacher et al., Circ. Res., 101:59-68, 2007.
Kuhl et al., Cell, 50:1057, 1987.
Kunz et al., Nucl. Acids Res., 17:1121, 1989.
Kutryk and Stewart, Microsc. Res. Tech., 60:138-158, 2003.
Lagos-Quintana et al., Curr. Biol., 12:735-739, 2002.
Lagos-Quintana et al., Science, 294(5543):853-858, 2001.
Landgraf et al., Cell, 129:1401-1414, 2007.
LaPointe et al., Hypertension 27(3 Pt 2):715-22, 1996.
LaPointe et al., J. Biol. Chem., 263(19):9075-8, 1988.
Larsen et al., Proc Natl. Acad. Sci. USA., 83:8283, 1986.
Laspia et al., Cell, 59:283, 1989.
Latimer et al., Mol. Cell. Biol., 10:760, 1990.
Latronico et al., Circ. Res., 101:1225-1236, 2007.
Lau et al., Science, 294(5543):858-862, 2001.
Le Gal La Salle et al., Science, 259:988-990, 1993.
Lee and Ambros, Science, 294(5543):862-864, 2001.
Lee et al., Nature, 294:228, 1981.
Lelievre et al., Bull. Cancer, 88:137-142, 2001.
Levinson et al., Nature, 295:79, 1982.
Levrero et al., Gene, 101:195-202, 1991.
Lin et al., Mol. Cell. Biol., 10:850, 1990.
Liu et al., Proc Natl Acad Sci USA 101:9740-9744, 2004.
Lowes et al., J. Clin. Invest., 100(9):2315-2324, 1997.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Mann et al., Cell, 33:153-159, 1983.
Marelli-Berg et al., J. Immunol. Methods, 244:205-215, 2000.
Markowitz et al., J. Virol., 62:1120-1124, 1988.
McNeall et al., Gene, 76:81, 1989.
Meister and Tuschl, Nature, 431:343-9, 2004.
Miksicek et al., Cell, 46:203, 1986.
Miyoshi et al., Oncogene, 23:5567-5576, 2004.
Mordacq and Linzer, Genes and Dev., 3:760, 1989.
Moreau et al., Nucl. Acids Res., 9:6047, 1981.
Moss et al., Biol. Chem., 271(49):31688-31694, 1996.
Muesing et al., Cell, 48:691, 1987.
Musiyenko et al., J. Mol. Med., 86:313-322, 2008.
Ng et al., Nuc. Acids Res., 17:601, 1989.
Nicolas and Rubinstein, In: Vectors: A survey of molecular cloning vectors and their uses, Rodriguez and Denhardt (Eds), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nonami et al., J. Biol. Chem., 279:52543-52551, 2004.
Omitz et al., Mol. Cell. Biol., 7:3466, 1987.
Ondek et al., EMBO J., 6:1017, 1987.
Palmiter et al., Nature, 300:611, 1982.
Panka et al., Clin. Cancer Res., 12:2371s-2375s, 2006.
Parker et al., Nature, 428:754-758, 2004.
Paskind et al., Virology, 67:242-248, 1975.
Pasquinelli and Ruvkun, Ann. Rev. Cell Dev. Biol., 18:495-513, 2002.
PCT Appln. WO 0071096
PCT Appln. WO 84/03564
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Physicians Desk Reference
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe, et al., Mol. Cell. Biol., 10:689-695, 1990.
Rittling et al., Nuc. Acids Res., 17:1619, 1989.
Rosen et al., Cell, 41:813, 1988.
Rosenfeld et al., Science, 252:431-434, 1991.
Rosenfeld, et al., Cell, 68:143-155, 1992.
Roux et al., Proc. Natl. Acad. Sci. USA, 86:9079-9083, 1989.
Sakai et al., Genes and Dev., 2:1144, 1988.
Sambrook and Russell, Molecular Cloning: A Laboratory Manual 3^{rd} Ed., Cold Spring Harbor Laboratory Press, 2001.
Satake et al., J. Virology, 62:970, 1988.
Schaffner et al., J. Mol. Biol., 201:81, 1988.
Scheinowitz et al., Int. J. Cardiol., 59:1-5, 1997.
Schmidt et al., Development, 134:2913-2923, 2007.
Searle et al., Mol. Cell. Biol., 5:1480, 1985.
Semenza, Annu. Rev. Med., 54:17-28, 2003.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Soncin et al., EMBO J., 22:5700-5711, 2003.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Stuart et al., Nature, 317:828, 1985.
Suarez et al., Circ. Res., 100:1164-1173, 2007.
Sullivan and Peterlin, Mol. Cell. Biol., 7:3315, 1987.
Swartzendruber and Lehman, J. Cell. Physiology, 85:179, 1975.
Syed et al., Cardiology, 101:131-143, 2004.
Takahashi et al., Nat. Med., 5:434-438, 1999.
Takebe et al., Mol. Cell. Biol., 8:466, 1988.
Tavazoie et al., Nature, 451:147-152, 2008.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, Eleventh Edition
Thiesen et al., J. Virology, 62:614, 1988.
Top et al., J. Infect. Dis., 124:155-160, 1971.
Treisman, Cell, 46(4):567-174, 1986
Tronche et al., Mol. Biol. Med., 7:173, 1990.
Tronche et al., Mol. Cell. Biol., 9(11):4759-4766, 1989.
Trudel and Constantini, Genes and Dev., 6:954, 1987.
Tur-Kaspa et al., Mol. Cell Biol., 6:716-718, 1986.
Tyndell et al., Nuc. Acids. Res., 9:6231, 1981.
van Rooij and Olson, J. Clin. Invest., 117:2369-2376, 2007.
van Rooij et al., Circ. Res., 94:e18-26, 2004.
van Rooij et al., Proc. Natl. Acad. Sci. USA, 103(48):18255-18260, 2006.
van Rooij et al., Science 316(5824):575-579. 2007
Vannice and Levinson, J. Virology, 62:1305, 1988.
Varmus et al., Cell, 25:23-36, 1981.
Vasseur et al., Proc Natl. Acad. Sci. USA, 77:1068, 1980.
Wagner et al., Proc. Natl. Acad. Sci. USA 87(9):3410-3414, 1990.
Wakioka et al., Nature, 412:647-651, 2001.
Wang and Calame, Cell, 47:241, 1986.
Wang et al., Proc. Natl. Acad. Sci. USA, 105:7738-7743, 2008.
Weber et al., Cell, 36:983, 1984.
Weinberger et al. Mol. Cell. Biol., 8:988, 1984.
Wienholds et al., Science, 309:310-311.
Winoto and Baltimore, Cell, 59:649, 1989.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Xu et al., Curr. Biol., 13:790-795, 2003.
Yamauchi-Takihara et. al., Proc. Natl. Acad. Sci. USA, 86(10):3504-3508, 1989.
Yang and Russell, Proc. Natl. Acad. Sci. USA, 87:4144-4148, 1990.
Yang et al., Nat. Med. 13:486-491, 2007.
Yutzey et al. Mol. Cell. Biol., 9:1397, 1989.
Zelenin et al., FEBS Lett., 287(1-2):118-120, 1991.
Zeng et al., Cancer Res., 62(13):3630-3635, 2002.
Ziober and Kramer, J. Bio. Chem., 271(37):22915-22, 1996.

1. A method of promoting vascular integrity and/or vascular repair comprising administering to a subject at risk of or suffering from vascular damage an agonist of miR-126 function.

2. The method of claim 1, wherein said subject is suffering from vascular damage.

3. The method of claim 2, wherein said vascular damage is to a cardiac tissue.

4. The method of claim 2, wherein said vascular damage comprises an ischemic event.

5. The method of claim 4, wherein said ischemic event comprises an infarct, ischemia-reperfusion injury or arterial stenosis.

6. The method of claim 2, wherein said vascular damage is in a non-cardiac tissue.

7. The method of claim 6, wherein said vascular damage comprises trauma or vascular leakage.

8. The method of claim 1, wherein said subject is at risk of vascular damage.

9. The method of claim 8, wherein said subject suffers from hypertension, late stage atherosclerosis cardiac hypertrophy, osteoporosis, neurodegeneration, fibrosis or respiratory distress.

10. The method of claim 1, wherein said subject is a non-human animal.

11. The method of claim 1, wherein said subject is a human.

12. The method of claim 1, wherein said agonist is miR-126.

13. The method of claim 1, wherein said agonist is a mimetic of miR-126.

14. The method of claim 1, wherein said agonist is an expression vector comprising an miR-126-encoding nucleic acid segment under the control of a promoter active in a target cell.

15-19. (canceled)

20. The method of claim 1, further comprising administering to said subject a secondary therapy.

21. The method of claim 1, wherein administering comprises systemic administration.

22-25. (canceled)

26. A method of inhibiting pathologic vascularization in a subject in need thereof comprising administering to the subject at risk of or suffering from pathologic vascularization an antagonist of miR-126.

27. The method of claim 26, wherein said subject is suffering from pathologic vascularization.

28. The method of claim 27, wherein said pathologic vascularization comprises early stage atherosclerosis, retinopathy, cancer or stroke.

29. The method of claim 26, wherein said subject is at risk of pathologic vascularization.

30. The method of claim 29, wherein said subject suffers from hyperlipidemia, obesity, asthma, arthritis, psoriasis and/or blindness.

31. The method of claim 26, wherein said subject is a non-human animal.

32. The method of claim 26, wherein said subject is a human.

33. The method of claim 26, wherein said antagonist is a miR-126 antagomir.

34. (canceled)

35. The method of claim 26, further comprising administering to said subject a secondary anti-angiogenic therapy.

36-40. (canceled)


SEQUENCE LISTING (14 sequences)

SEQ ID NO: 1 - RNA, 22 nt, Artificial
  Synthetic primer
ucguaccgugaguaauaaugcg

SEQ ID NO: 2 - RNA, 60 nt, Mus musculus
cauuauuacuuuugguacgcgcugugacacuucaaacucuguaccgugaguaauaaugcg

SEQ ID NO: 3 - DNA, 32 bp, Artificial
  Synthetic primer
acacttcctgtttcctggcccagaggaaacag

SEQ ID NO: 4 - DNA, 32 bp, Artificial
  Synthetic primer
acacttcctgtttcctggctcagaggaaacag

SEQ ID NO: 5 - DNA, 32 bp, Artificial
  Synthetic primer
tcacttcctgtttcctggctgagaggaaacag

SEQ ID NO: 6 - DNA, 32 bp, Artificial
  Synthetic primer
tcacttcctgtttcctggcttaggggaaacag

SEQ ID NO: 7 - DNA, 25 bp, Artificial
  Synthetic primer
cacaggaagtccggcgggaagggcc

SEQ ID NO: 8 - DNA, 25 bp, Artificial
  Synthetic primer
cacaggaagtccggctggaattgtc

SEQ ID NO: 9 - RNA, 22 nt, Artificial
  Synthetic primer
gcguaauaaugagugccaugcu

SEQ ID NO: 10 - RNA, 30 nt, Artificial
  Synthetic primer
guuuauauuuaacuaaauguaagguacgaa

SEQ ID NO: 11 - RNA, 30 nt, Artificial
  Synthetic primer
guuuauauuuaacuaaauauaagguacgaa

SEQ ID NO: 12 - RNA, 30 nt, Artificial
  Synthetic primer
guuuauauuuaacuaaguguaagguacgaa

SEQ ID NO: 13 - RNA, 31 nt, Artificial
  Synthetic primer
guuuauauuuaacuaaauaauaagguacgaa

SEQ ID NO: 14 - RNA, 30 nt, Artificial
  Synthetic primer
gucuauauuuaacuaaauauacgguccgca